(19) United States
(12) Patent Application Publication
(10) Pub. No.: US 2018/0282367 A1
(43) Pub. Date: Oct. 4, 2018

(54) NEW METHODS FOR MAKING BARUSIBAN AND ITS INTERMEDIATES

(71) Applicant: Ferring B.V., Hoofddorp (NL)

(72) Inventors: Leila Malik, Copenhagen S (DK); Kazimierz Wisniewski, San Diego, CA (US); Chantal Devin, Strasbourg (FR)

(21) Appl. No.: 15/765,933

(22) PCT Filed: Oct. 6, 2016
(86) PCT No.: PCT/EP2016/073858

§ 371 Date: Apr. 4, 2018

(30) Foreign Application Priority Data
Oct. 6, 2015 (EP) 15188529.0

(51) Int. Cl. C07K 1/04; C07K 7/16; C07K 7/06

(52) U.S. Cl. CPC C07K 1/042; C07K 7/06; C07K 7/16

(57) ABSTRACT

The present invention relates to new solid phase peptide methods for synthesizing analogues that exhibit oxytocin antagonist activity, specifically Barusiban and its intermediates. Specifically, the present invention relates to a solid phase process for preparing a compound having the formula c[AA₁-AA₆]-AA₇-ol, wherein AA₁ is propionic acid, AA₂ is preferably D-Trp, AA₃ is Ile, AA₄ is preferably AlloIle, AA₅ is Asn, AA₆ is hCy, and AA₇ is preferably N-Me-Orn-ol, or a pharmaceutically acceptable salt or solvate thereof.

### FIELD OF THE INVENTION

The present invention relates to new solid phase peptide methods for synthesizing analogues that exhibit oxytocin antagonist activity, specifically Barusiban and its intermediates, and that are useful, inter alia, for decreasing or blocking uterus muscle contraction.

### BACKGROUND OF THE INVENTION

Oxytocin is a peptide hormone which stimulates contraction of the uterine muscles, and it is believed to be involved in the etiology of pre-term labor and dysmenorrhea. Oxytocin antagonists have proved to be useful in the control of these conditions, and oxytocin antagonist peptides of good potency and selectivity for therapeutic use are disclosed in WO 95/02609, published 26 Jan. 1995. They are often intended for administration in aqueous solution, and the manufacture of ready-for-use doses of such antagonists may require that such solutions be stable for extended periods; which they may not always be. The potential need to prepare such a medicament immediately prior to use was considered to be inconvenient and generated an improvement.

Barusiban is a synthetic cyclic heptapeptide containing five unnatural amino acids, one of which is a D-amino acid. Its CAS registry number is 285571-64-4 (of free base). The drug substance is chemically designated as C^{4,6},S¹-Cyclo(N-(3-sulfanylpropanoyl)-D-tryptophyl-L-isoleucyl-L-alloisoleucyl-L-asparaginyl-L-2-aminobutanoyl-N-methyl-L-ornithinol) and is represented by the chemical structure below:

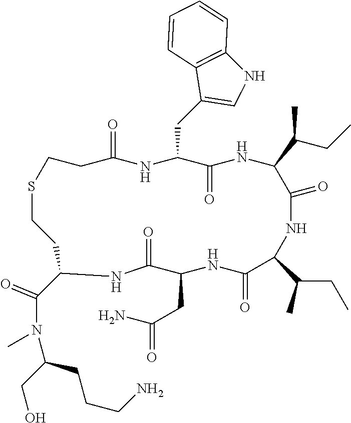 [SMILES: CC[C@@H](C)[C@@H]1NC(=O)[C@H]([C@@H](C)CC)NC(=O)[C@@H](Cc2c[nH]c3ccccc23)NC(=O)CCSCC[C@@H](C(=O)N(C)[C@H](CO)CCCN)NC(=O)[C@H](CC(N)=O)NC1=O]

The structure of Barusiban can also be represented as:

c[Pra-D-Trp-Ile-AlloIle-Asn-hCy]-N-Me-Orn-ol,

wherein Pra is propionic acid, Trp is tryptophan, Be is isoleucine, Allolle is alloisoleucine, Asn is asparagine, hCy is homocysteine and Orn is ornithine. “c” means that the sequence in brackets ([Pra-D-Trp-Ile-AlloIle-Asn-hCy]) is present in a cyclic form.

For the purposes of describing this invention, each amino acid in Barusiban will be given the shorthand notation as follows:

c[AA₁-AA₆]-AA₇-ol,

wherein AA₁ is propionic acid (pra), AA₂ is D-Trp, AA₃ is Be, AA₄ is AlloIle, AA_{S} is Asn, AA₆ is hCy, and AA₇ is N-Me-Orn-ol.

U.S. Pat. No. 6,143,722 (EP 938,496; WO 98/23636) discloses equivalent heptapeptide analogues that exhibit oxytocin antagonist activity, which resemble those disclosed in the earlier WO 95/02609 application, but wherein the C-terminus of the peptide is reduced to an alcohol.

Such oxytocin antagonist peptides can be synthesized by the synthesis disclosed in the U.S. Pat. No. 6,143,722. This requires about 7 separate steps, counting the peptide synthesis as one and not counting the synthesis of the modified homocysteine (hCy) residue.

An attempt to make a full SPPS method was outlined in WO2003072597. This document discloses a method for preparing a heptapeptide analogue, or a pharmaceutically acceptable salt thereof, having oxytocin antagonist activity and consisting of a hexapeptide moiety A and a C-terminal β-aminoalcohol residue B bound to the moiety A by an amide bond, wherein (1) the β-aminoalcohol B is:

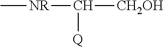

with Q being (CH₂)ₙ—NH₂, with n being 2, 3 or 4, and R being CH₃ or C₂H₅; and (2) the moiety A is:

Mpa-AA_{b}-Ile-AA_{d}-Asn-Abu-, and with AA_{b} being a D-aromatic α-amino acid, which may optionally have its side chain protected; and AA_{d} being an aliphatic α-amino acid.

These syntheses generally utilize such an alkylated diamino alcohol and the protected amino acid referred to as Fmoc-carba-6, i.e.,

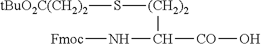

In this method, the cyclization is done in solution via an amide bond (coupling of Fmoc-hCy((CH₂)₂—COOBu)-OH) to AA_{b}, wherein AA_{b} may be D-Trp-OH). Accordingly, this approach requires the synthesis of orthogonally protected homo-cysteine derivatives (e.g. Fmoc-hCy((CH₂)₂—COOBu)-OH)), which may be tedious to manufacture in large scale.

Document CN 2012-1036484 discloses a process for preparation of Barusiban using SPPS. The process comprises the steps of (1) reacting Fmoc-N-Me-Orn(Boc)-ol with carboxy resin to obtain Fmoc-N-Me-Orn(Boc)-resin; (2) sequentially coupling of Fmoc-hCy(mmt)-OH, Fmoc-Asn(Trt)-OH, Fmoc-AlloIle-OH, Fmoc-Ile-OH, Fmoc-D-Trp(Boc)-OH and 3-halogenated propanoic acid to obtain X—CH₂CH₂CO-D-Trp(Boc)-Ile-AlloIle-Asn(Trt)-hCy(mmt)-N-Me-Orn(Boc)-resin; and (3) removal of the mmt side chain protecting group in order to perform the cyclization of the obtained linear peptide on the resin, followed by cleavage from the resin to obtain Barusiban.

Although such oxytocin antagonist peptides can be synthesized by the synthesis disclosed e.g. in U.S. Pat. No. 6,143,722, WO2003072597, CN102875650 and CN 2012-1036484, more economical synthesis, applicable in larger scales, with improved yield, better purity profile and/or more robust processes are frequently sought for chemical compounds of potential commercial interest. The present invention addresses this need.

### SUMMARY OF THE INVENTION

In general, this invention relates to a solid-phase synthesis of the heptapeptide Barusiban. Its CAS registry number is 285571-64-4 (of free base). It is chemically designated as C^{4,6},S¹-Cyclo(N-(3-sulfanylpropanoyl)-D-tryptophyl-L-isoleucyl-L-alloisoleucyl-L-asparaginyl-L-2-aminobutanoyl-N-methyl-L-ornithinol).

The present invention also provides heptapeptide analogues, or a pharmaceutically acceptable salt thereof, having oxytocin antagonist activity and new solid-phase peptide synthesis methods for preparing them.

The present invention relates to a solid phase process for preparing a compound having the formula c[AA₁-AA₆]-AA₇-ol, or a pharmaceutically acceptable salt or solvate thereof, wherein AA₁ is propionic acid, AA₂ is AA_{b}, AA₃ is Ile, AA₄ is AA_{d}, AA₅ is Asn, AA₆ is hCy, and AA₇ is —NR—CHQ-CH₂OH, wherein R is CH₃ or C₂H₅, preferably CH₃, Q is (CH₂)ₙ—NH₂, wherein n is 2, 3, or 4, preferably 3, the process comprising the steps of:
a) reacting protected (P₅;P₃)AA₇ with a resin to provide (P₅;P₃)AA₇- [symbol] wherein AA₇ as added to the resin during the synthesis in step a) is (P₅)NR-CHQ′-CH₂OH, wherein R is CH₃ or C₂H₅, Q′ is (CH₂)ₙ—N P₃ P₄;
b) stepwise lengthening (P₅;P₃)AA₇- [symbol] to provide (P₇)AA₂AA₃AA₄(P₁)AA₅(P₂)AA₆-(P₃)AA₇- [symbol] ;
c) reacting X-AA₁ (wherein X is an halogen atom selected from F, Cl, Br and I) with (P₇)AA₂AA₃AA₄(P₁)AA₅(P₂)AA₆-(P₃)AA₇- [symbol] to provide X-AA₁(P₇)AA₂AA₃AA₄(P₁)AA₅(P₂)AA₆-(P₃)AA₇- [symbol] ;
d) carrying out a cleavage and deprotection step to provide X-AA₁AA₂AA₃AA₄AA₅AA₆-AA₇-ol; and
e) cyclizing X-AA₁AA₂AA₃AA₄AA₅AA₆-AA₇-ol, obtaining the cyclic peptide:
c[AA₁-AA₆]-AA₇-ol wherein AA₁ and AA₆ are linked through a thiol from the AA₆ homocysteine,
wherein:
P₁, P₅, and P₇ are protecting groups,
AA_{b} is a D-aromatic α-amino acid;
AA_{d} is an aliphatic α-amino acid;
X is a halogen residue;
P₂ is Trt
R is CH₃ or C₂H₅;
Q′ is (CH₂)ₙ—NP₃P₄;
n is 2, 3, or 4;
P₃ and P₄ are independently H or an amino-acid protecting group, which may be the same or different from each other and which may the same or different to P₁ and/or P₂.

Particularly, the present invention relates to a solid phase process for preparing a compound having the formula c[AA₁-AA₆]-AA₇-ol, or a pharmaceutically acceptable salt or solvate thereof, wherein AA₁ is propionic acid, AA₂ is AA_{b}, preferably D-Trp, AA₃ is Ile, AA₄ is AA_{d}, preferably AlloIle, AA₅ is Asn, AA₆ is hCy; AA₇ is NR—CHQ-CH₂OH, wherein R is CH₃ or C₂H₅, Q is (CH₂)ₙ—NH₂, wherein n is 2, 3, or 4, wherein preferably, R is CH₃ and n is 3, wherein AA₇ is preferably N-Me-Orn-ol, the process comprising the steps of:

Stage 1a
a) reacting protected (P₅;P₃)AA₇ with a resin to provide (P₅;P₃)AA₇- [symbol] ; wherein AA₇ as added to the resin during the synthesis is (P₅)NR—CHQ′-CH₂OH, wherein R is CH₃ or C₂H₅, Q′ is (CH₂)ₙ—N P₃ P₄;
b) removal of P₅ from (P₅;P₃)AA₇- [symbol] to provide (P₃)AA₇- [symbol] ;

Stage 1b
c) reacting protected (P₆;Trt)AA₆ with (P₃)AA₇- [symbol] to provide (P₆;Trt)A₆-(P₃)AA₇- [symbol] ;
d) removal of P₆ from (P₆;Trt)AA₆-(P₃)AA₇- [symbol] to provide (Trt)AA₆-(P₃)AA₇- [symbol] ;
e) reacting protected (P₆;P₁)AA₅ with (Trt)AA₆-(P₃)AA₇- [symbol] to provide (P₆; P₁)AA₅(Trt)AA₆-(P₃)AA₇- [symbol] ;
f) removal of P₆ from (P₆;P₁)AA₅(Trt)AA₆-(P₃)AA₇- [symbol] to provide (P₁)AA₅(Trt)AA₆-(P₃)AA₇- [symbol] ;
g) reacting protected (P₆)AA₄ with (P₁)AA₅(Trt)AA₆-(P₃)AA₇- [symbol] to provide (P₆)AA₄(P₁)AA₅(Trt)AA₆-(P₃)AA₇- [symbol] ;
h) removal of P₆ from (P₆)AA₄(P₁)AA₅(Trt)AA₆-(P₃)AA₇- [symbol] to provide AA₄(P₁)AA₅(Trt)AA₆-(P₃)AA₇- [symbol] ;
i) reacting protected (P₆)AA₃ with AA₄(P₁)AA₅(Trt)AA₆-(P₃)AA₇- [symbol] to provide (P₆)AA₃AA₄(P₁)AA₅(Trt)AA₆-(P₃)AA₇- [symbol] ;
j) removal of P₆ from (P₆)AA₃AA₄(P₁)AA₅(Trt)AA₆-(P₃)AA₇- [symbol] to provide AA₃AA₄(P₁)AA₅(Trt)AA₆-(P₃)AA₇- [symbol] ;
k) reacting protected (P₆;P₇)AA₂ with AA₃AA₄(P₁)AA₅(Trt)AA₆-(P₃)AA₇- [symbol] to provide (P₆; P₇)AA₂AA₃AA₄(P₁)AA₅(Trt)AA₆-(P₃)AA₇- [symbol] ;

Stage 1c
l) removal of P₆ from (P₆;P₇)AA₂AA₃AA₄(P₁)AA₅(Trt)AA₆-(P₃)AA₇- [symbol] to provide (P₇)AA₂AA₃AA₄(P₁)AA₅(Trt)AA₆-(P₃)AA₇- [symbol] ;
m) reacting X-AA₁ (wherein X is an halogen atom selected from F, Cl, Br and I) with (P₇)AA₂AA₃AA₄(P₁)AA₅(Trt)AA₆-(P₃)AA₇- [symbol] to provide X-AA₁(P₇)AA₂AA₃AA₄(P₂)AA₅(Trt)AA₆-(P₃)AA₇- [symbol] ;

Stage 2
n) carrying out a cleavage and deprotection step to provide X-AA₁AA₂AA₃AA₄AA₅AA₆AA₇-ol; and

Stage 3
o) cyclizing X-AA₁AA₂AA₃AA₄AA₅AA₆AA₇-ol via thioether linkage through an intramolecular substitution of the halogen X with the AA₆ (homocysteine) thiol, obtaining the cyclic peptide:

c[AA₁-AA₆]-AA₇-ol,

wherein P₁, P₅, P₆ and P₇ are protecting groups, and wherein P₃ and P₄ are independently H or an amino-acid protecting group, which may be the same or different from each other and which may the same or different to P₁, and wherein n is 2, 3, or 4.

The present invention further relates to an intermediate suitable for forming a peptide having pharmaceutical properties, which has the formula:

X—CH₂CH₂CO-AA_{b}-Ile-AA_{d}-Asn(P₁)-hCy(P₂)-NR—CHQ′-CH₂OW

Wherein

AA_{b} is a D-aromatic α-amino acid;

AA_{d} is an aliphatic α-amino acid;

X is a halogen residue (F, Cl, Br, I);

P₁ is a protecting group

P₂ is a protecting group (Trt)

R is CH₃ or C₂H₅;

Q′ is (CH2)ₙ—NP₃P₄;

n is 2, 3, or 4;

P₃ and P₄ are independently H or an amino-acid protecting group, which may be the same or different from each other and which may the same or different to P₁ and/or P₂; and

W is H (namely, the C-terminus is an alcohol), a protecting group or a resin.

In one embodiment, X is not Cl when P₅ is Fmoc.

P₅ is preferably o-NBS (NBS).

The following abbreviations are used:
Abu 2-aminobutyric acid
AcOH acetic acid
Ac₂O acetic anhydride
AlloIle/aIle alloisoleucine
Asn asparagine
BH₃THF borane-tetrahydrofuran complex
Boc tert-butyloxycarbonyl
Br bromine
Br-(CH₂)₂—COOH 3-bromo propionic acid
CH₃CN acetonitrile
Cl chlorine
-Cl—(CH₂)₂—COOH 3-chloro propionic acid
CTC chlorotrityl chloride
DBU 1,8-diazabicyclo [5.4.0] undec-7-ene MeCN acetonitrile
DCM dichloromethane
DIAD diisopropyl azodicarboxylate
DIC 1,3-diisopropyl carbodiimide
DIEA N,N-diisopropylethylamine
DIPEA N,N-diisopropylethylamine
DME 1,2 dimethoxyethane
DMF N,N-dimethylformamide
DMAP 4-dimethylaminopyridine
DTT dithiothreitol
EDT ethanedithiol
-Et₂O ethyl ether
EtOAc ethyl acetate
Fmoc 9-fluorenylmethyloxycarbonyl
Fmoc-N-MeOrn(Boc)-ol methyl-N-α-(9-fluorenylmethoxy carbonyl)-N-δ-Boc-L-ornithol
Fmoc-hCy(Trt)-OH N-α-(9-fluorenylmethoxy carbonyl)-S-trityl-L-Homocysteine
Fmoc-hCy(mmt)-OH N-α-(9-fluorenylmethoxy carbonyl)-S-methoxytrityl-L-homocysteine
Fmoc-Asn(Trt)-OH N-α-(9-fluorenylmethoxy carbonyl)-N-β-trityl-L-Asparagine
Fmoc-aIle-OH N-α-(9-fluorenylmethoxy carbonyl)-N-L-Allolsoleucine
Fmoc-Ile-OH N-α-(9-fluorenylmethoxy carbonyl)-N-L-Isoleucine
Fmoc-D-Trp(Boc)-OH N-α-(9-fluorenylmethoxy carbonyl)-N(in)-Boc-D-Tryptophan
FmocONSu N-(9-fluorenylmethoxycarbonyloxy) succinimide
hCy/hCys/Hcy homocysteine
HOBt 1-hydroxybenzotriazole
HPLC high-performance liquid chromatography
H₂O deionized water
IPA isopropyl alcohol
LC/MS liquid chromatography-mass spectrometry
MeOH methanol
Mpa 3-mercaptopropionic acid
MTBD 7-methyl-1,5,7-triazabicyclo[4,4,0]dec-5-ene
MTBE methyl tert-butyl ether
Na₂CO₃ sodium carbonate
NMM N-methymorpholine
NMP 1-methylpyrrolidin-2-one
Orn ornithine
o-NBS (NBS) o-nitrobenzenesulfonyl
o-NBS-Cl o-nitrobenzenesulfonyl chloride
PhOH phenol
PyB OP benzotriazole-1-yl-oxy-tris-pyrrolidino phosphonium hexaflurophosphate
RRT relative retention time
SPPS solid-phase peptide synthesis
TBTU 2-(1-H-benzotriazol-1-yl)-1,1,3,3-tetramethyl-uronium tetrafluoroborate
TEAP triethylammonium phosphate
TFE trifluoroethanol
TFA trifluoroacetic acid
THF tetrahydrofuran
TIS triisopropylsilane
TNBS 2,4,6-trinitrobenzenesulfonic acid
TMSBr trimethylsilyl bromide
TPP triphenylphosphine
Trp tryptophan
Trt trityl
Z benzyloxycarbonyl

### BRIEF DESCRIPTION OF THE FIGURES

FIG. 1: Coupling of amino acids 2 to 7.

FIG. 2: Coupling of amino acid 1.

FIG. 3: Deprotection and cleavage of the heptapeptide from the resin to obtain the linear peptide.

FIG. 4: In solution cyclization of the peptide to obtain Barusiban.

FIG. 5: Purification of the cyclic peptide.

FIG. 6: Stability study of Fmoc-N-Me-L-Orn(Boc)-ol as a powder at different storage temperatures.

FIG. 7: Stability study of Fmoc-N-Me-L-Orn(Boc)-ol solution in DMF at different temperatures.

FIG. 8: Stability study of Fmoc-N-Me-L-Orn(Boc)-ol+DMAP, solution in DMF at room temperature.

FIG. 9: Stability study of Fmoc-N-Me-L-Orn(Boc)-ol+Pyridine, solution in DMF at 60° C.

FIG. 10: Stability study of o-NBS-N-Me-L-Orn(Boc)-ol+Pyridine solution in DMF at 60° C.

FIG. 11: Stability study of o-NBS-N-Me-L-Orn(Boc)-ol+DMAP solution in DMF at room temperature.

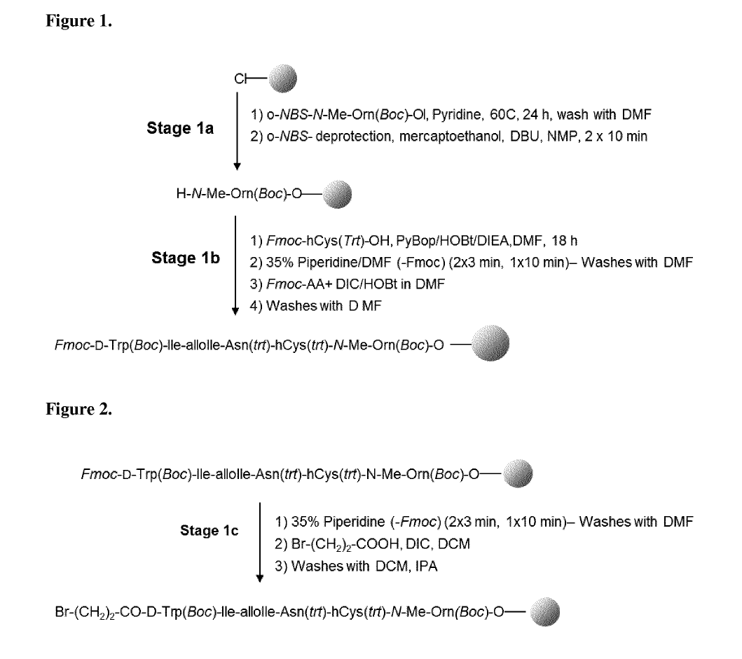

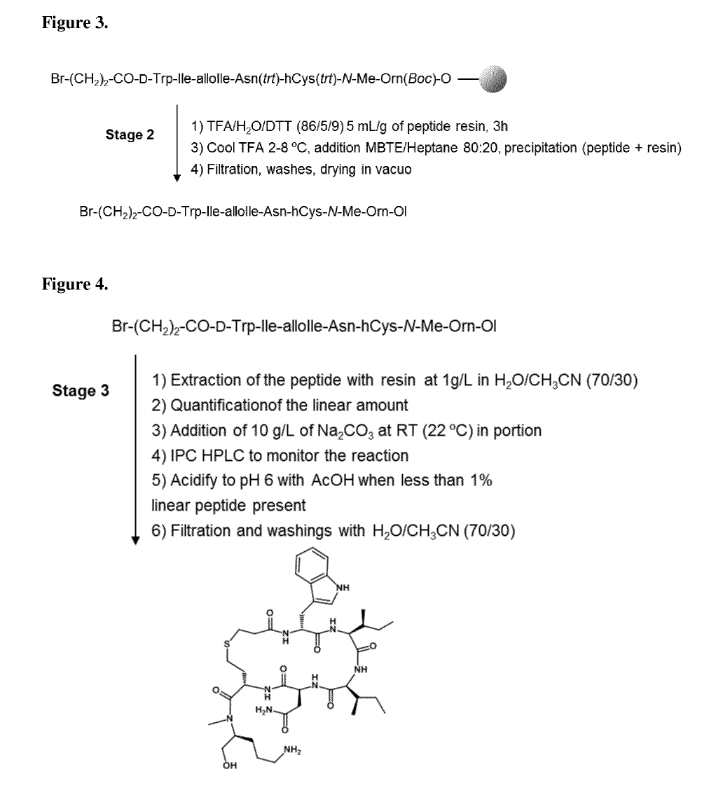

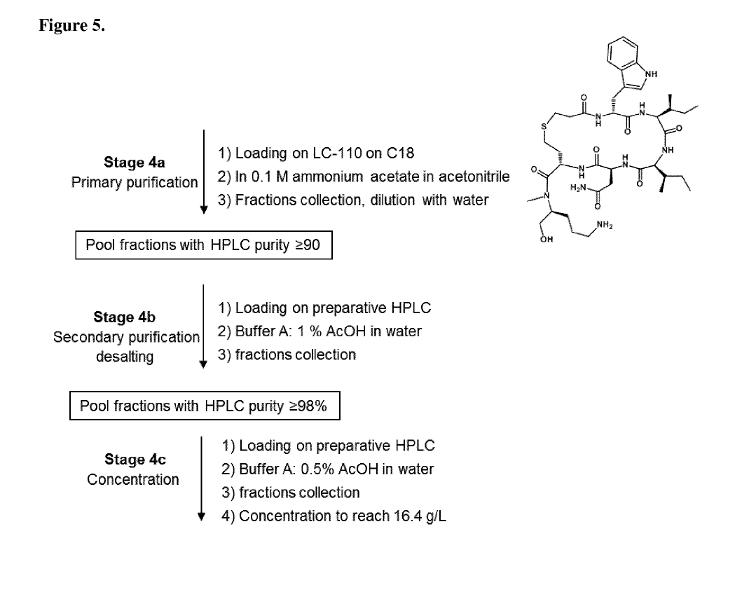

### DETAILED DESCRIPTION OF THE INVENTION

The present invention provides a solid phase process for preparing a cyclic heptapeptide analogue, preferably Barusiban, or a pharmaceutically acceptable salt thereof, having oxytocin antagonist activity comprising the steps of:

### Stage 1a

1. Attachment of P₅NR—CHQ′-CH₂OH (the first amino acid, AA₇), wherein P₅ is an amino acid protecting group, preferably NBS, to the resin. R is CH₃ or C₂H₅. Q′ is (CH₂)ₙ—NP₃P₄, where n is 2, 3, or 4 and P₃ and P₄ are independently H or an amino-acid protecting group, which may be the same or different from each other. Preferably, R is CH₃, n is 3, P₄ is H, P₃ is Boc and Pₛis o-NBS (NBS).

### The Solid Support

The solid support that can be used for the process of the present invention is not particularly limited, and essentially all solid supports that are used for solid phase peptide synthesis can be used for this process. In the present application, the solid support is also referred to as “-RESIN”. Exemplary solid supports are chloromethylated resins, hydroxymethylated resins, MBHA resin, BHA resin, NAMM resin, Rink amide AM resin, Rink amide MBHA resin, Rink amide MBHA resin, SASRIN resins, Sieber resins, Wang resins, super acid labile resins such as chlorotrityl resins. Particularly preferred are resins capable of forming an ether bond with an aliphatic alcohol, such as for example a trityl resin, a chlorotrityl resin (CTC) and/or a 2-CTC resin. Preferably, the resin is a chlorotrityl resin, even more preferably a 2-CTC resin. Preferably, the first amino acid to be attached to the resin (AA_{S}) is NBS-N-Me-Orn(P₃)-ol. Preferaby P₃ is Boc. The selection of this amino acid from other amino acids that could be used (e.g. Fmoc-N-Me-Orn(Boc)-ol) is due to its high stability profile, as it can be seen in the experimental part of this description.

Before addition of the first amino acid, the resin is deprotected, if needed.

The first amino acid is preferably charged to the (preferably deprotected) resin in an amount of approx. 0.5 to 1.5 equivalents, such as 0.5 equivalents, 0.6 equivalents, 0.7 equivalents, 1 equivalent, 1.3 equivalents or 1.5 equivalents. Preferably, the first amino acid (which is preferably NBS-N-Me-Orn(Boc)-ol) is charged in an amount approx. 0.7 equivalents to the (preferably deprotected) resin. Equivalents are molar equivalents.

Preferably, the first amino acid (preferably NBS—N-Me-Orn(Boc)-ol) is charged to the (preferably deprotected) resin in DMF. Preferably, pyridine is further added to the reaction mixture (resin+amino acid+DMF). For example, the reaction may take place at high temperatures, such as between 30 and 80° C., preferably at about 60° C. The reaction time may vary, but it may be between 2 and 30 h, such as between 10 and 25 h, such as between 15 and 18 h. Preferably, the reaction time is approximately 17 h (e.g. 17 h±15 min), or approximately 18 h. The reaction time may be approx. 24 h.

After the first amino acid has been loaded onto the resin, the remaining active sites may be capped for example by using DIEA/MeOH at room temperature during approximately one hour. The skilled person is aware of further capping methods.

2. Removing the protecting group (P₅, which is preferably o-NBS (NBS)) from the α-amino group of the first amino acid attached to the resin. The skilled person is aware of the deprotection conditions for each protecting group. For instance, Chem. Rev. 2009, 109, 2465-2504 (by Albert Isidro-Llobet et al.) provides a review on the protection of amino acids. In a preferred embodiment, where the first amino acid has NBS as α-amino protecting group, the removal of NBS is performed using β-mercaptoethanol and DBU in NMP (mercaptoethanol, DBU, NMP).

### Stage 1b

3. Adding the following amino acids using common Fmoc Solid Phase Peptide Synthesis protocols in this order:

P₆-hCy(Trt) (AA₆), P₆-Asn (AA₅), P₆-AA_{d} (AA₄), P₆-Ile (AA₃) and, finally, P₆-AA_{b} (AA₂)

AA_{b} is a D-aromatic α-amino acid, which may optionally have its side chain protected with P₇. P₇ is a protecting group (preferably Boc). AA_{d} is an aliphatic α-amino acid. Preferably, AA_{b} is D-Trp and AA_{d} is Allolle.

These amino acids (AA₆ to AA₂) are added to the resin with a free carboxyl group and a protected α-amino group (P₆), which may be the same or different, the preferred protecting group P₆ being Fmoc. The free carboxyl group is preferably pre-activated by subjecting the α-amino protected amino acid to peptide coupling agent and peptide coupling additive in an organic solvent. These protected amino acids are preferably charged in an amount of approx. 1 to 3 equivalents, such as approx. 1, approx. 1.5, approx. 2 or approx. 3 equivalents. For example, the protected amino acids AA₆, AA₅, AA₃ and AA₂ are charged in the resin in an amount of approx. 2 equivalents. For example, the protected amino acid AA₄ is charged in the resin in an amount of approx. 1.5 equivalents. For example, the coupling agents (e.g. DIC/HOBt) are charged in the resin in the same equivalents as the amino acids (e.g. approx. 2 equivalents for the coupling of AA₆, AA₅, AA₃ and AA₂ and e.g. approx. 1.5 equivalents for the coupling of AA₄).

As the skilled person knows, after coupling and/or deprotection of each amino acid, a coupling test may be carried out in order to verify that the coupling/deprotection has been completed. If the coupling/deprotection has not been completed, the reaction may be repeated (either coupling or deprotection). Examples of those tests are the “Kaiser test” (also known as “ninhydrin test”) (E. Kaiser, R. L. Colescott, C. D. Bossinger, P. I. Cook, Analytical Biochemistry 34 595 (1970)) or the “Chloranil test” (to test for free secondary amines, T Christensen, Acts Chemica Scandinavica B 33 (1979) 763-766).

The organic solvent, peptide coupling reagent, and peptide coupling additive may be any of those known in the art of solid phase peptide synthesis.

Typical organic solvents are THF, NMP, DCM, DMF, DMSO, IPA and mixtures thereof. The preferred solvents are NMP, DCM, and DMF or mixtures thereof. For example, the solvent of choice for the coupling reaction is DMF. For example, the solvent of choice for the deprotection reaction is DMF.

Typical peptide coupling reagents are one or more of o-(7-azabenzotriazol-1-yl)-1,1,3,3-tetramethyluronium hexafluorophosphate (HATU), o-(benzotriazol-1-yl)-1,1,3,3-tetramethyluronium hexafluorophosphate (HBTU), o-(benzotriazol-1-yl)-1,1,3,3-tetramethyluronium tetrafluoroborate (TBTU), benzotriazole-1-yl-oxy-tris(dimethylamino)phosphonium hexafluorophosphate (BOP), benzotriazole-1-yl-oxy-tris-pyrrolidinophosphonium hexafluorophosphate (PyB OP), N,N-bis-(2-oxo-3-oxazolidinyl)phosphonic dichloride (BOP-Cl), bromo-tris-pyrrolidino-phosphonium hexafluorophosphate (PyBroP), iso-butylchloroformate (IBCF), 1,3 dicyclohexylcarbodiimide (DCC), 1,3-diisopropyl-carbodiimide (DIC), 1-(dimethylaminopropyl)-3-ethylcarbodiimide hydrochloride (WSCDI), N-ethoxycarbonyl-2-ethoxy-1,2-dihydroquinoline (EEDQ), isopropylchloroformate (IPCF), 2-(5-norbornen-2,3-dicarboximido)-1,1,3,3-tetramethyluronium tetrafluoroborate (TNTU), propane phosphonic acid anhydride (PPAA), piridine and 2-succinimido-1,1,3,3-tetramethyluronium tetrafluoroborate (TSTU). Preferred coupling agents are DIC, PyBOP and HBTU.

Typical peptide coupling additives are 1-hydroxy-1H-benzotriazole (HOBt) and 1-hydroxy-7-azabenzotriazole (HOAt), preferably HOBt. In a particularly preferred embodiment, DIC and HOBt are used in combination. In another embodiment, PyBOP and HOBt may be used in combination.

Preferably, the second amino acid (P₆-hCy(P₂)-OH, preferably P₆-hCy(trt)-OH, more preferably Fmoc-hCy(Trt)-OH) is charged in an amount of approx. 1.5 equivalents or approx. 2 equivalents. Preferably, this coupling is performed in the presence of PyBOP. For example, this coupling is performed in the presence of PyBOP/HOBt/DIEA in DMF.

Preferably, the third coupling (P₆-Asn(P₁)-OH, preferably P₆-Asn(Trt)-OH, even more preferably Fmoc-Asn(Trt)-OH) is charged in an amount of approx. 2 equivalents. Preferably, this coupling is performed in the presence of DIC/HOBt, preferably in DMF, preferably in an amount of approx. 2 equivalents with respect to the incoming amino acid.

Preferably, the fourth coupling (P₆-AA_{d}-OH, preferably P₆-Allolle-OH, even more preferably Fmoc-Allolle-OH) is charged in an amount of approx. 1.5 equivalents. Preferably, this coupling is performed in the presence of DIC/HOBt, preferably in DMF, preferably in an amount of approx. 1.5 equivalents with respect to the incoming amino acid.

Preferably, the fifth coupling (P₆-Ile-OH, preferably Fmoc-Ile-OH) is charged in an amount of approx. 2 equivalents. Preferably, this coupling is performed in the presence of DIC/HOBt, preferably in DMF, preferably in an amount of approx. 2 equivalents.

Preferably, the sixth coupling (P₆-AA_{b}(P₇)-OH, preferably P₆-D-Trp(Boc)-OH, even more preferably Fmoc-D-Trp(Boc)-OH) is charged in an amount of approx. 2 equivalents. Preferably, this coupling is performed in the presence of DIC/HOBt, preferably in DMF, preferably in an amount of approx. 2 equivalents with respect to the incoming amino acid.

The peptide coupling agent is generally added in an amount of approx. 1.5 to 4, such as approx. 1.5, approx. 2, approx. 3, or approx. 4 equivalents, preferably approx. 1.5 or approx. 2 equivalents with respect to the incoming amino acid (equivalents are molar equivalents).

After each coupling step, the α-amino protecting group (P₆ for the second to the sixth coupling, which is preferably Fmoc) needs to be removed prior to the subsequent coupling step. Such a step is known in the art. If the α-amino protecting group P₆ is Fmoc, which is the preferred case, as stated above, the removal is preferably achieved by reaction with piperidine, DBU (1,8-diazabicyclo[5.4.0]-undec-7-ene), or DEA (diethylamine). The preferred solvent is DMF. Preferably, the removal of the Fmoc group is performed with a mixture of piperidine/DMF, preferably with approx. 35% piperidine in DMF (piperidine/DMF 35/65).

The following peptide-resin is created:

P₆-AA_{b}-Ile-AA_{d}-Asn(P₁)-hCy(Trt)-NR—CHQ′-CH₂—O-Resin

AA_{b} is a D-aromatic α-amino acid, preferably tryptophan (D-Trp).

AA_{d} is an aliphatic α-amino acid, preferably alloisoleucine (alloIle).

R is CH₃ or C₂H₅. Q′ is (CH₂)ₙ—NP₃P₄, where n is 2, 3, or 4 and P₃ and P₄ are independently H or an amino-acid protecting group, which may be the same or different from each other. Preferably, P₄ is H. Preferably, R is CH₃, n is 3, P₄ is H and P₃ is Boc. P₁, P₃ and P₇ may be the same or different. Preferably, P₃ and P₇ are the same protecting group, preferably Boc. Preferably, P₁ is Trt. Preferably, P₅ is NBS. P₆ is preferably orthogonal to P₁, P₃ and P₇. Barany et al. (Barany, G.; Merrifield, R. B. J. Am. Chem. Soc. 1977, 99, 7363; Barany, G.; Albericio, F. J. Am. Chem. Soc. 1985, 107, 4936) described the concept of orthogonality, in the sense that the two or more protecting groups belong to independent classes and are removed by distinct mechanisms. Accordingly, these protecting groups (P₁, P₃ and P₇) are selected such that they are not removed in the step of removing the α-amino protecting group (P₅ and P₆), and preferably such that they can be removed together in a single reaction step, most preferably in the step of cleaving the peptide from the resin. Most preferred for P₁ is Trt, and for P₃ and P₇, Boc. P₆ is preferably Fmoc.

The following table lists preferred protecting groups for —NH₂ and —OH, together with preferred cleaving reagents.

TABLE 1
Side chain protecting groups and cleavage conditions (Isidro-
Llobet et al., Chem. Rev. 2009, 109, 2465-2504)

| Protecting group | | Protected | Cleavage | |
|---|---|---|---|---|
| Abbreviation | Name | group | reagent | Solvent |
| Cbz or Z | Benzyloxycarbonyl | —NH₂ | H₂/Pd—C | EtOH/Water/acid |
| | | | HF | Neat |
| | | | Trifluoromethane- | DCM |
| | | | sulfonic acid | |
| Boc | tert-Butoxy- | —NH₂ | TFA | DCM |
| | carbonyl | | HCl | Dioxane |
| | | | Methanesulfonic | DCM |
| | | | acid | |
| Trt | Trityl (Trt) | —OH | 1% TFA—DCM | DCM |
| | | —NH₂ | | |
| TBDMS | Tert-butyl- | —OH | TFA | THF |
| | dimethyl-silyl | | ACOH—THF—H₂O | |
| | | | (3:1:1), 18 h | |
| Cyclohexyl | Cyclohexyl | —OH | HF or TFSMA | Neat HF or |
| (CHX or CH_{X}) | | | | DCM |

### Stage 1c

4. Removing the amino acid protecting group P₆ and coupling the halogen-propionic acid (X-pra) (X—CH₂CH₂—COOH). X is a halogen residue selected from the group consisting of F,

Cl, Br and I. For example, X may be Cl or Br. Preferably, X is Br.

Preferably, this (seventh) coupling (halogen-propionic acid (X-pra), preferably 3-Br-propionic acid) is charged in an amount of approx. 3 equivalents. Preferably, this coupling is performed in the presence of DIC/DCM, preferably in an amount of approx. 3 equivalents with respect to the incoming amino acid.

As stated above, after coupling of the halogen-propionic acid (X-pra) (X—CH₂CH₂—COOH) (preferably Br—CH₂CH₂—COOH), a coupling test may be carried out in order to verify that the coupling has been completed. If the coupling has not been completed, the reaction may be repeated. Examples of those tests are the “Kaiser test” (also known as “Ninhydrin test”) or the “Chloranil test”. For example, after coupling of the halogen-propionic acid (X-pra) (X—CH₂CH₂—COOH) (preferably Br—CH₂CH₂—COOH), the Ninhydrin test may be performed in order to verify the completeness of the reaction.

After coupling of the halogen-propionic acid (X-pra) (X—CH₂CH₂—COOH) (preferably Br—CH₂CH₂—COOH), the peptide-resin may be washed with DCM and IPA.

The peptide coupling agent is generally added in an amount of approx. 1.5 to 4 (such as for example approx. 1.5, approx. 2 equivalents, or approx. 3 equivalents, or approx. 4 equivalents, preferably approx. 3 equivalents in the case of the seventh coupling, as described above) equivalents with respect to the incoming amino acid (equivalents are molar equivalents).

After the coupling of the halogen-propionic acid (X-pra) (X—CH₂CH₂—COOH) (preferably Br—CH₂CH₂—COOH), the following protected peptide is attached to the resin:

X-AA₁(P₇)AA₂AA₃AA₄(P₁)AA₅(Trt)AA₆-(P₃)AA₇-

[symbol]

preferably:

X—(CH₂)₂—CO-AA_{b}-Ile-AA_{d}-Asn(P₁)-hCy(Trt)-NR—CHQ′-CH₂O-

[symbol]

even more preferably,

Br—(CH₂)₂—CO-D-Trp(Boc)-Ile-alloIle-Asn(trt)-hCy(Trt)-N-Me-Orn(Boc)-O-

[symbol]

R is CH₃ or C₂H₅, Q′ is (CH₂)ₙ—NP₃P₄, where n is 2, 3, or 4 and P₃ and P₄ are independently H or an amino-acid protecting group, which may be the same or different from each other. X is a halogen residue, such as F, Cl, Br or I, preferably Br. AA_{b} is a D-aromatic α-amino acid, preferably tryptophan (D-trp). AA_{d} is an aliphatic α-amino acid, preferably alloisoleucine (alloIle). Preferably, R is CH₃, n is 3, P₄ is H and P₃ is Boc. Preferably, P₇ is Boc. Preferably, P₁ is Trt.

In one embodiment, X is not Cl when P₅ is Fmoc.

### Stage 2

5. Removing the side chain protecting groups (P₁, P₃, P₄ and P₇, if all of them are present) from the peptide and cleaving the peptide from the resin, obtaining the deprotected linear peptide:

X-AA₁AA₂AA₃AA₄AA₅AA₆AA₇-ol, preferably

X—(CH₂)₂—CO-AA_{b}-Ile-AA_{d}-Asn-hCy-NR—CHQ-CH₂OH, even more preferably

Br—(CH₂)₂—CO-D-Trp-Ile-alloIle-Asn-hCy-N-Me-Orn-ol

Wherein R is CH₃ or C₂H₅, Q is (CH₂)ₙ—NH₂, and n is 2, 3, or 4. Preferably, R is CH₃ and n is 3.

X is a halogen residue, such as Cl or Br, preferably Br.

AA_{b} is a D-aromatic α-amino acid, preferably tryptophan (D-trp).

AA_{d} is an aliphatic α-amino acid, preferably alloisoleucine (alloIle).

The cleavage conditions depend particularly on the type of resin and protecting groups used. According to the present invention, the cleavage is however preferably carried out under acidic conditions with TFA.

According to the present invention, these two steps (cleaving and removing the side chain protecting groups (P₁, P₃, P₄ and P₇, if they are all present) are preferably combined in a single deprotection and cleavage step.

Preferably, the cleavage and deprotection is performed in a single step in the presence of a cleavage cocktail comprising (or, alternatively, consisting of) TFA, H₂O and DTT, preferably in a ratio of approx. 86/5/9 (TFA/H₂O/DTT), preferably in an amount of approx. 5 mL (of cleavage cocktail/g of peptide resin), during approx. 1-5 h, preferably during approx. 3 h.

For example, the cleavage may be performed at room temperature, such as approx. 25° C. The cleavage may be performed at a temperature of 15-30° C., such as approx. 15° C., approx. 20° C. or approx. 25° C., or approx. 30° C. Preferably, the cleavage is performed at room temperature (RT).

After cleavage, the deprotected peptide and resin may be precipitated, for example by cooling the TFA (e.g. −5 to 8° C., such as 2 to 8° C., preferably at a temperature of 0° C.±5° C.) and adding e.g. MBTE/n-Heptane, preferably in a ratio of 80:20, or preferably in a ratio of 70:30 (MBTE:n-Heptane). Preferably, the temperature is controlled so that it remains under approx. 20° C.

After cleavage, the deprotected peptide may be extracted from the peptide-resin with e.g. water/CH₃CN: 7/3, 1.6 g/L. After cleavage, the crude linear peptide and the resin may be filtered off and washed e.g. with MTBE (for example three times in an amount of approx. 3 mL per gram of peptide-resin) and dried under vacuum.

6. Optionally purifying the linear peptide. Purification may be performed by chromatographic techniques such as preparative reverse phase chromatography (RPC).

The deprotected linear peptide (optionally purified) is thus obtained:

X-AA₁AA₂AA₃AA₄AA₅AA₆AA₇-ol,

preferably

X—(CH₂)₂—CO-AA_{b}-Ile-AA_{d}-Asn-hCy-NR—CHQ-CH₂-OH,

more preferably

Br—(CH₂)₂—CO-D-Trp-Ile-alloIle-Asn-hCy-N-Me-Orn-ol.

R is CH₃ or C₂H₅, Q is (CH₂)ₙ—NH₂, and n is 2, 3, or 4. Preferably, R is CH₃ and preferably n is 3. X is a halogen residue, such as F, Cl, Br or I, preferably Br. AA_{b} is a D-aromatic α-amino acid, preferably tryptophan (D-Trp). AA_{d} is an aliphatic α-amino acid, preferably alloisoleucine (alloIle).

### Stage 3

7. Performing the cyclization of the peptide in solution through an intramolecular substitution of the halogen X (preferably Br) with the homocysteine (hCy) thiol. Preferably, the cyclization occurs in the presence of a suitable base, such as a carbonate (e.g., sodium carbonate, potassium carbonate) or organic bases such as DIEA, followed by acidification (e.g., pH 5.5 or pH 6) with a suitable acid (e.g., acetic acid). Preferably, the cyclization occurs in the presence of sodium carbonate (Na₂CO₃).

To a 1 and 2 g of peptide/L of solution, preferably to a concentration of approx. 1 g of peptide/L solution), Na₂CO₃ (for example in an amount of approx. 10 g/L) is added. The cyclization reaction may be monitored by HPLC, as the skilled person knows.

Once the cyclization has been completed, the pH of the solution may be adjusted to e.g. 5.5 or pH 6 by addition of a suitable base, for example AcOH. The cyclic peptide may then be filtrated and washed with water/CH₃CN (7/3).

The cyclic heptapeptide analogue, preferably Barusiban, or a pharmaceutically acceptable salt thereof, having oxytocin antagonist activity is thus obtained: c[(CH₂)₂—CO-AA_{b}-Ile-AA_{d}-Asn-hCy]-NR—CHQ-CH₂OH, preferably c[(CH₂)₂—CO-D-Trp-Ile-AlloIle-Asn-hCy]-N-MeOrn-ol, also represented as

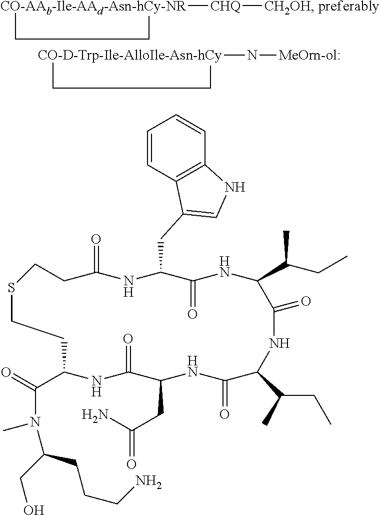

R is CH₃ or C₂H₅, Q is (CH₂)ₙ—NH₂, and n is 2, 3, or 4. Preferably, R is CH₃ and preferably n is 3. X is a halogen residue, such as F, Cl, Br or I, preferably Br. AA_{b} is a D-aromatic α-amino acid, preferably tryptophan (D-Trp). AA_{d} is an aliphatic α-amino acid, preferably alloisoleucine (alloIle).

Optionally, the cyclic peptide can be purified. Purification may be performed by chromatographic techniques such as preparative reverse phase chromatography (RPC). Subsequent to purification, the peptide can be lyophilized.

In addition, the invention provides an intermediate peptide which has the formula:

X—(CH₂)₂—CO-AA_{b}-Ile-AA_{d}-Asn(P₂)-hCy(P₂)—NR—CHQ′-CH₂OW

Wherein AA_{b} is a D-aromatic α-amino acid (preferably D-Trp), which may optionally have its side chain protected, AA_{d} is an aliphatic α-amino acid (preferably allolle), X is a halogen residue selected from the group consisting of F, Cl, Br and I, preferably Br, P₁ is a protecting group (preferably Trt), P₂ is Trt, R is CH₃ or C₂H₅, Q′ is (CH₂)ₙ-NP₃P₄, where n is 2, 3, or 4 and P₃ and P₄ are independently H or an amino-acid protecting group, which may be the same or different from each other and which may the same or different to P₁ and/or P₂, and wherein W is H (namely, the C-terminus is an alcohol), a protecting group or a resin. Preferably P₄ is H and P₃ is Boc. Preferably, R is CH₃, n is 3, P₄ is H and P₃ is Boc.

Preferably, AA_{b} is D-Trp, preferably with the side chain protected with a protecting group P₇. Preferably, P₇ is Boc. Preferably, AA_{d} is AlloIle.

In addition, the invention provides an intermediate peptide which has the formula:

X—(CH₂)₂—CO-D-Trp(Boc)-Ile-AlloIle-Asn(Trt)-hCy(Trt)-N-Me-Orn(Boc)-OW,

wherein X is a halogen residue selected from the group consisting of F, Cl, Br and I, preferably Br and wherein W is H (namely, the C-terminus is an alcohol), a protecting group or a resin. Preferably, the invention provides an intermediate peptide which has the formula:

X—(CH₂)₂—CO-D-Trp-Ile-AlloIle-Asn-hCy-N-Me-Orn-ol,

wherein X is a halogen residue selected from the group consisting of F, Cl, Br and I, preferably Br.

In addition, the invention provides an intermediate peptide which has the formula:

Br—(CH₂)₂—CO-D-Trp-Ile-AlloIle-Asn-hCy-N-Me-Orn-ol.

As used herein, the terms “about” and “approx.” mean the indicated value ±1% of its value, or the terms “about” and “approx.” mean the indicated value ±2% of its value, or the terms “about” and “approx.” mean the indicated value ±5% of its value, the terms “about” and “approx.” mean the indicated value ±10% of its value, or the terms “about” and “approx.” mean the indicated value ±20% of its value, or the terms “about” and “approx.” mean the indicated value ±30% of its value; preferably the terms “about” and “approx.” mean exactly the indicated value (±0%).

### 1. EXAMPLES

### Example 1: Synthesis of Barusiban Synthesis Flow Chart

### 1.2. Description of the Manufacturing Process

### Assembly

The protected o-NBS-N-MeOrn(Boc)-ol is coupled directly onto the chloro-2-chlorotrityl resin (CTC resin) in DMF at 60° C. in presence of pyridine during 17 hours. After capping of the resin, the NBS group is deprotected by washes with a DBU/mercaptoethanol/NMP mixture. After removal of the NBS protecting group, the second protected amino acid (Fmoc-hCy(Trt)-OH) is coupled with PyBOP/HOBt/DIEA in DMF. The reaction completion is checked by a coupling test (Chloranil test). The four other residues (Fmoc-Asn(Trt)-OH, Fmoc-Allolle-OH, Fmoc-Ile-OH and Fmoc-D-Trp(Boc)-OH) are incorporated by a succession of Fmoc deprotection and amino acid coupling cycles:
1. Fmoc removal
2. DMF washes
3. Couplings of Fmoc-AA-OH with DIC/HOBt in DMF.
4. Coupling test (Ninhydrin test or Chloranil test)
5. DMF washes

Reactions volumes are calculated on the basis of 5 mL/g of peptide-resin. Deprotection of the Fmoc protecting group occurs in piperidine (35% in DMF) by three repeated cycles (3 min, 3 min and 10 min). Washings are performed with DMF by seven repeated cycles after deprotection of the Fmoc protecting group, and by three repeated cycles after the coupling step. All coupling reactions are carried out with 2 eq of Fmoc protected amino acid and 2 eq of DIC/HOBt in DMF excepted for Fmoc-alloIle-OH coupling (1.5 eq instead of 2 eq). After each amino acid coupling reaction completeness is controlled by a semi-quantitative colour test based on revealing the unreacted amines. Primary amines are tested by the Ninhydrin test (Kaiser test) (Kaiser et al., Analytical Biochem., 39, 305, (1975); E. Kaiser, R. L. Colescott, C. D. Bossinger, P. I. Cook, Analytical Biochem., 34 (Issue 2), 595-598, (1970)) and secondary amines with Chloranil test (Kaiser et al., Analytical Biochem., 39, 305, (1975)). The last coupling reaction is done with 3 eq of 3-bromopropionic acid and 3 eq of DIC in solution in DCM to obtain the protected linear peptide.

### Cleavage:

After the assembly step, cleavage from the resin and concomitant side chain deprotection is performed in a TFA mixture leading to the crude linear peptide. The peptide is cleaved from the resin and deprotected with TFA/H₂O/DTT mixture (86/5/9 v/v/w) at a concentration of 5 mL per gram of peptide-resin during 3 h 00 at room temperature. The peptide-resin is added portion wise by controlling the temperature (T≤20° C.). The reaction mixture is cooled at 0° C.±5° C. and a mixture MTBE/n-Heptane (7/3; 50 mL per gram of peptide-resin) is poured onto the reaction mixture to precipitate the cleaved linear peptide within the resin under temperature control (T<20° C.). The crude linear peptide+resin is filtered off, washed with MTBE (3×3 mL per gram of peptide-resin) and dried under vacuum.

### Cyclization:

The linear peptide is dissolved in a solution of water/CH₃CN (7/3) at a concentration of 1.6 g/L. Then the solution is adjusted at a concentration of 1 g/L and is stirred overnight in order to complete the decarboxylation of the peptide. Before the cyclization, the amount of linear peptide in solution is estimated by HPLC via a reference sample of linear peptide at a concentration of 1 g/L. The peptide is cyclized at a concentration of 1 g/L of peptide with the resin and in presence of sodium carbonate (10 g/L). The completeness of the cyclization is monitored by HPLC. Once the cyclization is completed (less than 1% of linear peptide), the pH of the reaction mixture is decreased to around 5.5 by acetic acid addition. The resin is filtered off and washed with water/CH₃CN (7/3 v/v). Then the filtrate solution is diluted by half with water and stored at 2-8° C.

### 2. Example 2: Synthesis of Crude Barusiban Using 3 Chloropropionic Acid Instead of 3 Bromopropionic Acid

An experiment was performed according to the process described above (see Example 1) where the 3-bromopropionic acid is replaced by 3-chloropropionic acid.

### 2.1. Assembly

Resin loading

The assembly was performed on 5 mmoles scale (7.14 g of chloro-2-chlorotrityl resin (CTC resin) with a substitution of 0.7 meq/g). After swelling of the resin in DMF (7 mL) during 15 minutes, o-NBS—N-MeOrn(Boc)-ol (1 eq, 2.92 g) was dissolved in 9 mL of DMF and added onto the resin. Pyridine (2 eq, 0.81 mL) was added and the reaction mixture was heated to 60° C. and stirred during 17 hours. After 1 h, 6 mL of DMF were added in order to homogenize the reaction mixture. The concentration of o-NBS—N-MeOrn(Boc)-ol during the loading reaction was 0.22 mol/L. After 17 h, 15 mL of DMF were added onto the resin in order to homogenize the reaction mixture before capping. The stability of the o-NBS—NMeOrn(Boc)-ol in those conditions has been checked; the study is described in Example 5. It appears that the material is stable without any degradation in these loading conditions.

### Resin Capping

DIEA (5.7 eq, 4.96 mL) and MeOH (4.96 mL, v/v) were added onto the resin and stirred for 1 hour at room temperature in order to neutralize the unreacted sites of the resin. The resin is then drained and washed with DMF (3×45 mL).

### NBS Deprotection and Fmoc-hCy(Trt)-OH Coupling

The NBS protecting group was removed by two repeated cycles (2×10 min) with a mixture of DBU/Mercaptoethanol (5 eq/10 eq, 3.74 mL/3.51 mL) in NMP (30 mL). The second amino acid (Fmoc-hCy(Trt)-OH) was coupled with PyBOP/HOBt/DIEA (1.5 eq/1.5 eq/3.75 eq; 1.15 g/3.90 g/3.26 mL) in DMF (23 mL). After 16 hours, the reaction completion was checked by a Chloranil test and the resin was washed with DMF (3×51 mL).

### Fmoc Deprotection:

The following amino acids (Fmoc-Asn(Trt)-OH, Fmoc-alloIle-OH, Fmoc-Ile-OH and Fmoc-D-Trp(Boc)-OH) were incorporated by a succession of Fmoc deprotection and amino acid coupling cycles according to the manufacturing process described above (Example 1). The Fmoc protecting group was removed by three repeated cycles (3, 3 and 10 min) with a mixture of piperidine 35% in DMF. After the Fmoc deprotection, the washings were performed with DMF by seven repeated cycles and after the coupling step with DMF by three repeated cycles. Fmoc deprotection volumes are calculated on the basis of 5 mL/g of peptide-resin

### Fmoc-AA-OH Coupling:

For Fmoc-Asn(Trt)-OH, Fmoc-Ile-OH and Fmoc-D-Trp(Boc)-OH), 2 eq. of amino acid were coupled in presence of 2 eq of DIC/HOBt in DMF (25 mL). For Fmoc-alloIle —OH, 1.5 eq. of amino acid were coupled in presence of 1.5 eq of DIC/HOBt in DMF (25 mL). For all couplings, the reaction completion was checked by a Ninhydrin test and the resin was washed with DMF by three repeated cycles.

### 3 chloropropionic Acid Coupling

The last coupling was performed by dissolving 1.63 g of the 3-chloropropionic acid (3 eq) and 2.34 mL of DIC in DCM (42.5 mL). The reaction completion was checked by a Ninhydrin test (coupling time 32 2 hours). At the end of the assembly, the resin was washed with DCM (3×77 mL), IPA (5×77 mL) and dried under vacuum. 12 g of protected peptide-resin are obtained which represents a recovery yield of 92.4%.

### Cleavage and Deprotection

12 g of protected peptide-resin were added portion wise onto a TFA/H₂O/DTT mixture (86/5/9 v/v/w) (60 mL) by controlling the temperature (T≤20° C.). The reaction mixture was stirred at room temperature during 3 h 00. The reaction mixture was then cooled down to 2.7° C. and a mixture MTBE/n-Heptane (7/3 v/v) (600 mL) was poured into the reactor by maintaining the temperature below 20° C. The crude linear peptide+resin was filtered off, washed with MTBE (3×36 mL) and dried under vacuum overnight. 13 g of the crude chloro linear peptide+resin are obtained. The HPLC purity is 76,6%.

### 2.2. Cyclization

The total amount of chloro linear peptide+resin is divided in two portions and the cyclization is performed twice on around 6.5 g.

### 2.2.1. First Cyclization

A first cyclization was performed on 6.5 g according to the process described in Example 1 above. The cyclization did not go to completion, as analyzed by HPLC. Consequently, a second cyclization was initiated on the remaining approx. 6.5 g of chloro linear peptide.

### 2.2.2. Second Cyclization

The second cyclization was performed on 6.44 g of the chloro linear peptide+resin. The chloro linear peptide+resin was dissolved in 595 mL of a water/CH₃CN (7/3 v/v) solution giving the peptide in solution at a concentration of 1 g/L. 5.95 g of solid sodium carbonate were added (pH 11.3). The reaction was regularly monitored by HPLC). After 25 h, HPLC monitoring showed less than 1% of linear peptide. 8.35 mL of acetic acid were added to neutralize the reaction mixture; the resulting pH of the solution was 5.6. After acidification, the resin was removed by filtration and washed twice with 30 mL of water/CH₃CN (7/3) mixture. The filtrate solution was then diluted with 660 mL of water and stored at 2-8° C. The final concentration of the crude peptide in solution was 0.45 g/L with a purity of 86.4% by HPLC. The results obtained from both assemblies are compared in order evaluate the differences between using 3-bromo propionic acid (Example 1) and 3-chloro propionic acid (Example 2) in the synthesis process of Barusiban as described in Examples 1 and 2.

### Assembly Results

Following table (Table 2) summarizes and compares the assembly data obtained on experiment of Example 2 and production of Example 1:

TABLE 2
Comparative table of assembly data for experiment
of Example 1 and production of Example 2.

| | Example 1 | | Example 2 | |
|---|---|---|---|---|
| Synthesis scale | 3000 | mmoles | 5 | mmoles |
| Coupling time of the first 2 AA | | | | |
| o-NBS-N-MeOrn-(Boc)-ol | 17 h 00 | | 17 h 00 | |
| Fmoc-hCys(Trt)-OH | 19 h 30 | | 16 h 00 | |
| Coupling time of the other AA | | | | |
| Fmoc-Asn(Trt)-OH | 2 h 15 | | 2 h 10 | |
| Fmoc-aIle-OH | 2 h 00 | | 2 h 30 | |
| Fmoc-Ile-OH | 3 h 00 | | 3 h 25 | |
| Fmoc-D-Trp(Boc)-OH | 3 h 00 | | 2 h 15 | |
| 3-halopropionic acid | 1 h 15 | (bromo) | 2 h 00 | (chloro) |
| (last coupling) | | | | |
| Assembly yield | | | | |
| With loading correction | 72% | | 65% | |
| | 89.8% | | 92.4% | |
| | 8421.7 | g | 13.0 | g |

| HPLC purity of linear peptide | quantitative | quantitative | |
|---|---|---|---|
| | 74.64% | 76.6% | |
| Time of the cyclization step | 1 h-1 h 30 | 25 | h |

| | | |
|---|---|---|
| HPLC purity of crude peptide | 85.24% | 86.36% |

Until coupling of the 3-halopropionic acid, both assemblies are performed according to the same conditions; consequently it is not surprising to observe similar coupling times. Furthermore, no significant difference in the coupling time is observed by changing the last reagent; 3-chloropropionic acid (experiment of Example 2) shows similar coupling time than 3-bromopropionic acid (Example 1), both reactions are complete in less than 2 hours. By taking into account the loading of the resin, both assemblies show a comparable yield (around 90%). A major difference lies in the reaction time needed for cyclization; indeed, the cyclization is complete after 1 hour in the process using the bromo derivative (Example 1) whereas 25 hours are required by using the chloro derivative (Example 2). The bromo linear peptide shows a better reactivity compared to the chloro linear peptide.

HPLC purity of the cyclic crudes are similar; 86.36% for 3-chloropropionic acid and 85.24% for 3-bromopropionic acid.

These assembly results show that Barusiban can be synthesized from 3-chloropropionic acid but the cyclization time is longer than by using the 3-bromopropionic acid, as shown in Examples 1 and 2.

### HPLC Impurity Profile of Crude Barusiban

The crude Barusiban solutions synthesized whether from 3-chloropropionic acid or from 3-bromopropionic acid were analyzed by HPLC and LC/MS in order to identify the impurities and to compare the impurities profiles.

The major impurities are listed in the Table 3 below:

TABLE 3
Comparative table of impurities for experiments
of Example 1 and Example 2.

| | Experiment (Bromo) | Experiment (Chloro) |
|---|---|---|
| | Example 1 | Example 2 |
| | (1^{st} cyclization) | (2^{nd} cyclization) |
| HPLC purity of crude | — | 86.36% |
| | 85.24% | |
| Major impurities | | |
| by LC/MS (System III) | | |
| RRT = 0.23 | 0.05% (−227; −Ile | 0.17% |
| | or −aIle; -Trp) | |
| RRT = 0.54 | 0.19% (H-D-Trp- | 0.17% (H-D-Trp- |
| | (. . .)-N-MeOrn-ol) | (. . .)-N-MeOrn-ol) |
| RRT = 0.68 | — | 0.36% (+84) |
| RRT = 0.74 | 0.19% (+90) | 0.15% (+90; +138; |
| | | double oxydation) |
| RRT = 0.75 | 0.58% | — |
| RRT = 0.79 | 0.21% | — |
| | (same mass) | |
| RRT = 0.82 | 0.03% | 0.40% |
| | | (same mass) |
| RRT = 0.84 | 0.06% | 1.30% (linear peptide + |
| | | 2 oxydations) |
| RRT = 0.85 | 0.56% | — |
| RRT = 0.89 | — | 0.10% |
| | | (deamidation) |
| RRT = 0.91 | 0.15% | — |
| | (+tBu; +71) | |
| RRT = 0.94 | — | 0.13% (+CO₂ |
| | | adduct from Boc) |
| RRT = 0.98 | 0.35% (+72) | — |
| RRT = 1.05 | — | 1.32% |
| | | (deamidation; +41; −Ile |
| | | or −aIle) |
| RRT = 1.11 | — | 0.13% (+tBu; linear |
| | | peptide - Asn-Hcys) |
| RRT = 1.12 | 0.43% (+117) | — |
| RRT = 1.16 | — | 2.06% |
| | | (+tBu; linear peptide) |
| RRT = 1.17 | 0.50% (+117) | — |
| RRT = 1.19 | — | 1.79% |
| | | (−Asn; +tBu) |
| RRT = 1.22 | 0.58% | — |
| | (−Asn; dimer) | |
| RRT = 1.25 | — | 2.96% (−Asn) |
| RRT = 1.26 | 3.69% | 1.87% (−Asn) |
| | (+194; +tBu; +136) | |
| RRT = 1.30 | 1.81% (+194) | — |
| RRT = 1.32 | 3.80% (−Asn) | — |
| RRT = 1.35 | — | 0.19% |
| RRT = 1.38 | 0.03% | 0.18% |
| RRT = 1.42 | 0.14% | — |
| RRT = 1.65 | 0.10% | — |
| RRT = 1.68 | 0.29% | — |
| RRT = 1.74 | 0.29% | — |

The manufacturing process of the invention (e.g. Example 1) using the 3-bromopropionic acid is more adapted since the cyclization reaction is faster and generates less side reactions.

### Conclusion of Examples 1 and 2

The aim of experiment of Example 2 was to evaluate the replacement of 3-bromo propionic acid by 3-chloro propionic acid in the Barusiban's synthesis process as described in Example 1. Based on all results obtained after these trials (see Table 2 and Table 3), use of 3-chloropropionic acid is possible; nevertheless the cyclization reaction is longer due to a lower reactivity of the chloro linear peptide. The prolonged reaction time involves partial deamidation of the peptide in the aqueous basic solution. The impurities formed might be difficult to separate in the purification step.

### 3. Example 3. Synthesis of Barusiban Following a Different Synthetic Approach

### 3.1. Synthesis Flow Chart

The flow chart of the manufacture of Barusiban according to the process of Example 3 is presented below:

### 3.2. Description of the Manufacturing Process

### 3.2.1. Assembly

The protected Fmoc-N-MeOrn(Boc)-ol is coupled directly onto the carboxylic resin in DMF at room temperature in presence of DMPA during 2 hours. After capping of the resin, the

Fmoc group is deprotected by washes with a piperidine solution (20% in DMF). The five other residues (Fmoc-hCy(mmt)-OH, Fmoc-Asn(Trt)-OH, Fmoc-alloIle-OH, Fmoc-Ile-OH and Fmoc-D-Trp(Boc)-OH) are incorporated by succession of Fmoc deprotection and amino acid coupling cycles:
1. Fmoc removal
2. DMF washes
3. Coupling of Fmoc-AA-OH with DIC/HOBt in DMF by pre-activation of the amino acid
4. Coupling test (Ninhydrin test or Chloranil test)
5. DMF washes

Reaction volumes are calculated on the basis of 5 mL/g of peptide-resin. Deprotection of the Fmoc protecting group occurs in piperidine (20% in DMF) by two repeated cycles (10 min and 20 min). Washings are performed with DMF by seven repeated cycles after Fmoc deprotection, and by three repeated cycles after the coupling step. Pre-activation is performed with 3eq of Fmoc-AA-OH and 3.3 eq of DIC/HOBt in DMF by stirring during 5 minutes at 0° C. After pre-activation, all coupling reactions are carried out for 2 hours at room temperature. Because this is a critical step, after each amino acid coupling reaction, completeness is controlled by a semi-quantitative color test based on revealing the unreacted amines. Primary amines are tested by the Ninhydrin test (Kaiser Test), and secondary amines with Chloranil test. The last coupling is done with 3-halopropionic acid in solution in DMF to obtain the protected linear peptide.

### 3.2.2. Deprotection of Homo-Cysteine and Cyclization

After the assembly step, the mmt protecting group of the homo-Cysteine (hCy) is deprotected and the peptide-resin is cyclized on the resin. Reaction volumes are calculated on the basis of 5 mL/g of peptide-resin. The peptide-resin is washed with DCM by three repeated cycles and the mmt group is removed by five repeated cycles with a mixture of TFA/EDT/DCM (2/3/95 v/v/v). After the deprotection, washings are performed with DCM and DMF by three repeated cycles. The peptide is cyclized onto the resin with a solution of tetramethylguanidine (1% in DMF) during 8 hours at room temperature. After cyclization, the cyclic peptide-resin is washed with DMF, DCM, and MeOH before drying under vacuum.

### 3.2.3. Cleavage

After the cyclization, cleavage from the resin and concomitant side chain deprotection is performed in a TFA mixture leading to the crude Barusiban. The peptide is cleaved from the resin and deprotected with TFA/TIS/EDT/PhOH/H₂O mixture (90/3/2/1/4 v/v/v/v/v) at a concentration of 10.6 mL per gram of peptide-resin during 2 hours at room temperature. The reaction mixture is filtered off and the resin is washed with TFA. The reaction mixture is poured onto dry Et₂O (106 mL/g of peptide-resin) to precipitate the crude cyclic peptide. The precipitate is filtered off, washed with dry Et₂O (3×3.3 mL per gram of peptide-resin) and dried under vacuum.

### 3.3. Experiment Plan

In order to compare Barusiban's synthesis following different coupling times, it was decided to split the experiments in two pathways:
A first pathway in which the couplings are stopped after 2 hours, even if the coupling test is positive (Example 3.1).
A second pathway in which the couplings are performed during 2 hours minimum and stopped only after completeness by Kaiser test; in this case the coupling times might be different (IPC) (Example 3.2).

In addition, for each experiment (3.1 and 3.2), a further variable was introduced, namely the last amino acid coupled being 3-bromo propionic acid or 3-chloro propionic acid (see scheme below). There are in total 4 different syntheses:
Experiment 3.1.1: couplings stopped after 2 hours; 3-chloro propionic acid
Experiment 3.1.2: couplings stopped after 2 hours; 3-bromo propionic acid
Experiment 3.2.1: couplings performed during 2 hours minimum and stopped only after completeness by Kaiser test; 3-chloro propionic acid

Experiment 3.2.2: couplings performed during 2 hours minimum and stopped only after completeness by Kaiser test; 3-bromo propionic acid

Following scheme (Scheme 1) summarizes the experiment plan followed for the synthesis of crude Barusiban according to Example 3 (3.1.1, 3.1.2, 3.2.1 and 3.2.2):

Loading of the resin is performed on 2 mmoles of carboxylic resin with a substitution of 1.9 mmol/g. After the loading step the quantity is split in 2 equal portions (1 mmoles each) and the assembly is continued whether by using the first pathway (experiment 3.1) or the second pathway (experiment 3.2). After coupling of the Fmoc-D-Trp(Boc)-OH, the quantity is split in 2 equal portions (0.5 mmoles each) and the assembly is continued for each pathway whether by using the 3-chloropropionic acid (experiments 3.1.1 and 3.2.1) or the 3-bromopropionic acid (experiments 3.1.2 and 3.2.2).

### 3.4. Synthesis of Fmoc-N-MeOrn(Boc)-ol

Fmoc-N-MeOrn(Boc)-ol is used as starting material in the processes described in Example 3. This derivative is not the same as the one used in the process described in Examples 1 and 2, thus it was synthesized before the assembly. The Fmoc derivative was obtained from the material o-NBS-N-MeOrn(Boc)-ol used in Examples 1 and 2 in a two steps procedure described below.

### 3.4.1. Reaction Scheme

Fmoc-N-MeOrn(Boc)-ol is synthesized from o-NBS—N-MeOrn(Boc)-ol in two steps according to the reaction scheme described below (Scheme 2).

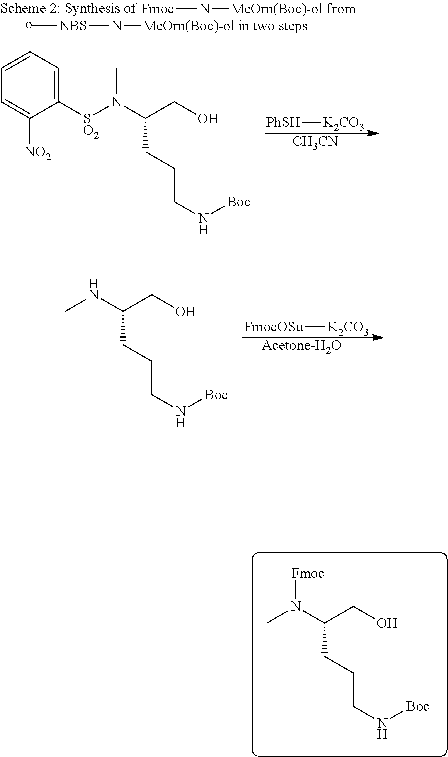

### 3.4.2. Step 1: Deprotection of o-NBS Group by PhSH

The o-NBS group is deprotected by a treatment with thiophenol in basic solution. After concentration of acetonitrile and addition of acidic water, major organic impurities are removed by washes with IPE.

### Experimental:

o-NBS—N-Me-L-Orn(Boc)-ol (50 g, leq, 119.8 mmol) was dissolved in CH₃CN (16V) in a 3 L necked flask. K₂CO₃ (54.6 g, 3.5 eq, 395.3 mmol) and PhSH (30.5 mL/33g, 2.5 eq, 299.4 mmol) were added to the reaction mixture and stirred at room temperature. The reaction completion was checked by TLC (AcOEt/Cycloheaxane/AcOH, 5/5/0.5 and CHCl₃/Methanol/AcOH, 60/10/5). After 19 h, the solvent was removed by concentration on a rotary evaporator and water (800 mL) was added. The aqueous phase was washed twice with IPE (2×300 mL) and the solution was used as such in the next step.

### 3.4.3. Step 2: Fmoc Protection with Fmoc-OSu

The aqueous phase is used as such and Fmoc protection is performed by using Fmoc-OSu in acetone/water. After acidification of the reaction mixture, the solution is extracted by AcOEt and washed with basic and acid solutions.

### Experimental:

Fmoc-OSu (36.4 g, 0.9 eq, 107.8 mmol) in acetone (800 mL) was added to the reaction mixture containing H—N-Me-L-Orn(Boc)-ol and stirred at room temperature overnight. The reaction completion was checked by TLC (AcOEt/Cycloheaxane/AcOH, 5/5/0.5 and CHCl₃/Methanol/AcOH, 60/10/5). After overnight, AcOEt (800 mL) was added and the aqueous phase was discarded. The organic phase was washed twice with a solution NaHCO₃ (5% in water) (2×400 mL), twice with a solution KHSO₄ 1N (2×400 mL), and once with water (400 mL) and brine (400 mL). The organic phase was then dried over Na₂SO₄. The compound is purified by flash chromatography using silica gel and AcOEt/Cyclohexane as eluent. After concentration of the pure fractions, Fmoc-N-MeOrn(Boc)-ol is isolated as a white foam.

### Experimental:

After filtration and concentration on a rotary evaporator, the crude product was purified by flash chromatography on silica gel (Eluent: AcOEt/Cyclohexane 3/7 (5 L) then 7/3 (10 L)). The pure fractions were concentrated, co-evaporated with cyclohexane (300 mL) and dried under vacuum (<100 mmHg) during 20h to give a white wax. Cold pentane (−20° C.) was added and the slurry mixture was stirred during 1 h at −20° C. Filtration was not possible as the Fmoc-N-MeOrn(Boc)-ol did not form a solid. Pentane was removed by concentration on rotary evaporator and the white foam was dried under vacuum (<100 mmHg) during 20 h. 34.1g of Fmoc-N-MeOrn(Boc)-ol were obtained corresponding to a yield of 70% on the two steps. The HPLC purity of the Fmoc derivative is 99.10%. Stability of the Fmoc-N-MeOrn(Boc)-ol was checked in various conditions and is described in Example 5.

### 3.5. Synthesis of Crude Barusiban According to the Process of Example 3

### 3.5.1. Loading and Capping

Resin loading:

The loading was performed on 2 mmol of carboxylic resin with a substitution of 1.9 meq/g.

The carboxylic resin (1 eq, 1.05 g) was swollen in DMF (5.26 mL) during 30 minutes and drained. In parallel, Fmoc-N-MeOrn(Boc)-ol (2 eq, 1.82g) was dissolved in DMF (3.4 mL) and pre-activated with DIC (leq, 0.31 mL) during 5 minutes at 0° C. After the pre-activation, Fmoc-N-MeOrn(Boc)-ol was added to the resin. DMAP (0.22 eq, 54 mg) was added and the mixture stirred for 2 hours at room temperature. After 2 h, the resin was drained and washed with DMF (3×7.6 mL). The stability of the Fmoc-N-MeOrn(Boc)-ol in those conditions is described in Example 5, as stated above. It appears that partial degradation is already observed after 2 hours at room temperature; the material can potentially decompose during the loading reaction.

### Resin Capping:

DMF (3.4 mL) was added in the reactor followed by the addition of DIEA (0.83 eq, 290 μL) and MeOH (1.7 mL). The reaction mixture was stirred for 1 h 30 at room temperature in order to neutralize the unreacted sites of the resin. The resin was then drained and washed with DMF (3×7.6 mL). According to the experiment plan described above (scheme I), the total amount of peptide-resin was divided in two portions. The first portion was used for the synthesis through pathway 1 (first pathway, coupling time 2 h, Example 3.1) and the second portion for the synthesis through pathway 2 (the second pathway, variable coupling times (minimum 2h) depending on completeness by Kaiser test Example 3.2).

### 3.5.2. First Pathway: Experiment According to Example 3.1

For the first pathway, the five following amino acids were assembled on 1 mmoles scale up to the peptide Fmoc-D-Trp(Boc)-Ile-allolle-Asn(Trt)-hCy(mmt)-N-MeOrn(Boc)-O-Resin

### Fmoc Deprotection:

The following amino acids (Fmoc-hCy(mmt)-OH, Fmoc-Asn(Trt)-OH, Fmoc-alloIle-OH, Fmoc-Ile-OH and Fmoc-D-Trp(Boc)-OH) were incorporated by a succession of Fmoc deprotection and amino acid coupling cycles according to the manufacturing process of

Example 3 as described above (see section 3.2). The Fmoc protecting group was removed by two repeated cycles (10 and 20 min) with a mixture of piperidine 20% in DMF. After the deprotection, the washings were performed with DMF by seven repeated cycles and after the coupling step with DMF by three repeated cycles. Fmoc deprotection reaction volumes are calculated on the basis of 5 mL/g of peptide-resin.

### Fmoc-AA-OH Coupling:

3 eq of the following amino acids (Fmoc-hCy(mmt)-OH, Fmoc-Asn(Trt)-OH, Fmoc-alloIle-OH, Fmoc-Ile-OH and Fmoc-D-Trp(Boc)-OH) were pre-activated with 3.3 eq of DIC/HOBt in DMF at 0° C. during 5 minutes before addition onto the resin in the reactor. All couplings were performed during 2 hours and checked by a Chloranil or Ninhydrin test. Coupling tests were negative for Fmoc-Asn(Trt)-OH, Fmoc-alloIle-OH, Fmoc-Ile-OH and Fmoc-D-Trp(Boc)-OH. Despite a positive test on the Fmoc-hCy(mmt)-OH coupling, the reaction was stopped (after 2 h). After coupling test, the resin was washed with DMF by three repeated cycles. After coupling of Fmoc-D-Trp(Boc)-OH, the total amount of peptide-resin was divided in two portions and the coupling of 3-halopropionic acid (either Cl, Example 3.1.1 or Br, Example 3.1.2) was performed on 0.5 mmoles scale.

### 3 Halopropionic Acid Coupling: Experiments According to Examples 3.1.1 and 3.1.2

Two experiments were carried out in parallel for the last coupling. An experiment was performed with the 3-chloropropionic acid (Example 3.1.1) and another experiment was conducted with the 3-bromopropionic acid (Example 3.1.2). These couplings were performed within 2 hours at room temperature by pre-activating the 3-halopropionic acid (3 eq) with DIC/HOBt (3.3 eq) in DMF during 5 minutes at 0° C. After 2 hours, the reaction completion was checked by a Ninhydrin and TNBS test:
For the bromo derivative, both tests were positive
For the chloro derivative, Ninhydrin test showed light blue beads and a positive TNBS test.

Despite these positive coupling tests, the coupling reactions were stopped. The resin was then washed with DMF (3×5 mL).

### Deprotection of Homo-Cysteine and Cyclization

The peptide-resin was washed with DCM (3×5 mL) and the mmt group was deprotected by five repeated cycles (5×10 min) with 4 mL of a TFA/EDT/DCM (2/3/95 v/v/v) mixture. After the deprotection, washings were performed with DCM (3×5 mL) and DMF (3×5 mL).

Cyclization was performed on the resin by adding 5 mL of tetramethylguanidine solution (1% in DMF) and stirring during 8 hours at room temperature. After cyclization, the cyclic peptide-resin was washed with DMF (3×6 mL), DCM (3×6 mL), MeOH (5×6 mL) and dried under vacuum. 278 mg of the cyclic peptide-resin are obtained on experiment according to Example 3.1.1 by using the 3-chloropropionic acid and 292 mg on experiment according to Example 3.1.2 by using the 3-bromopropionic acid corresponding respectively to an assembly yield of 8.3% and 13.3% by taking into account the loading.

### Cleavage

Cleavage from the resin was performed for each peptide according to the general conditions described in section 3.1.

### Experiment with 3-chloropropionic acid: Example 3.1.1

278 mg of protected cyclic peptide-resin were suspended in a TFA/TIS/EDT/PhOH/H₂O (90/3/2/1/4) (v/v/v/v/v) mixture (2.95 mL) and the reaction mixture was stirred at room temperature during 2 hours. The reaction mixture was then filtered off and the resin was washed with TFA (195 μL). The TFA mixture was poured onto dry Et₂O (29.5 mL); contrary to expectation, no precipitation of the crude peptide was obtained. The mixture containing the crude peptide was concentrated to dryness on a rotary evaporator. The concentration residue was dissolved in H₂O/CH₃CN (50/50 v/v) and freeze-dried. After freeze-drying, a very low quantity of powder was obtained (approx. 1-3 mg) with a purity of 11.8%.

### Experiment with the 3-bromopropionic acid: Example 3.1.2

272 mg of protected cyclic peptide-resin were suspended in a TFA/TIS/EDT/PhOH/H₂O (90/3/2/1/4) (v/v/v/v/v) mixture (3,1 mL) and the reaction mixture was stirred at room temperature during 2 hours. The reaction mixture was then filtered off and the resin was washed with TFA (204 μL). The TFA mixture was poured onto dry Et₂O (31 mL); as for the experiment with 3-chloropropionic acid, no precipitation of the crude peptide was obtained. The mixture containing the crude peptide was concentrated to dryness on a rotary evaporator. The concentration residue was dissolved in H₂O/CH₃CN (50/50) (50/50 v/v) and freeze-dried.

After freeze-drying, a very low quantity of the powder was obtained (approx. 1-3 mg) with a purity of 1.8%.

### 3.5.3. Second pathway: Experiment 3.2

As explained previously in section 3.3, the second pathway corresponds to the synthesis according to the process of Example 3 where all coupling reactions are controlled by IPC. In this case, the couplings are performed during 2 hours minimum and stopped only after reaching a negative coupling test. Two experiments were carried out according to the second pathway: one experiment with 3-chloro propionic acid (experiment according to Example 3.2.1) and another with 3-bromo propionic acid (experiment according to Example 3.2.2).

### Resin Loading: Experiment 3

The resin loading is described in section 3.5.1 (common loading for the two pathways). After the loading test, the total amount of peptide-resin was divided in two portions. For the second pathway (Example 3.2), the five following amino acids were assembled on 1 mmoles scale up to the peptide Fmoc-D-Trp(Boc)-Ile-allolle-Asn(Trt)-hCy(mmt)-N-MeOrn(Boc)-O-Resin (Experiment according to section 3.2).

### Fmoc Deprotection:

The following amino acids (Fmoc-hCy(mmt)-OH, Fmoc-Asn(Trt)-OH, Fmoc-alloIle-OH, Fmoc-Ile-OH and Fmoc-D-Trp(Boc)-OH) were incorporated by a succession of Fmoc deprotection and amino acid coupling cycles according to the manufacturing process described above (see section 3.2, Example 3). The Fmoc protecting group was removed by two repeated cycles (10 and 20 min) with a mixture of piperidine 20% in DMF. After deprotection, the washings were performed with DMF by seven repeated cycles and after the coupling step with DMF by three repeated cycles. Fmoc deprotection reaction volumes are calculated on the basis of 5 mL/g of peptide-resin.

### Fmoc-AA-OH Coupling:

3 eq of the following amino acids (Fmoc-hCy(mmt)-OH, Fmoc-Asn(Trt)-OH, Fmoc-alloIle-OH, Fmoc-Ile-OH and Fmoc-D-Trp(Boc)-OH) were pre-activated with 3.3 eq of DIC/HOBt in DMF at 0° C. during 5 minutes before addition onto the resin in the reactor. For all couplings, the reaction completion was checked by a Ninhydrin or Chloranil test. Coupling tests were negative for Fmoc-Asn(Trt)-OH, Fmoc-alloIle-OH, Fmoc-Ile-OH and Fmoc-D-Trp(Boc)-OH after 1 hours and the stirring was continued during an additional hour to reach 2 hours of coupling time. Fmoc-hCy(mmt)-OH coupling required 4 hours to reach a negative coupling test. After coupling test, the resin was washed with DMF by three repeated cycles. After coupling of Fmoc-D-Trp(Boc)-OH, the total amount of peptide-resin was divided in two portions and the coupling of 3-halopropionic acid was performed on 0.5 mmoles scale.

### 3 Halopropionic Acid Coupling: Experiments According to Example 3.2.1 and Example 3.2.2

Two experiments were carried out in parallel for the last coupling. An experiment was performed with the 3-chloropropionic acid (3.2.1) and another experiment was conducted with the 3-bromopropionic acid (3.2.2). These couplings were performed at room temperature by pre-activating the 3-halopropionic acid (3 eq) with DIC/HOBt (3.3 eq) in DMF during 5 minutes at 0° C. The reaction completion was regularly checked by a Ninhydrin test and a TNBS test. After 21 h 15, the coupling test was still positive, thus 1.65 eq of DIC (128 μL) was added and the reaction mixture was stirred for additional 6 hours. After this period, no evolution was observed and the resin was drained.
Double coupling was performed by pre-activating 1.5 eq of the 3-halopropionic acid (3-chloropropionic acid or 3-bromopropionic acid) in presence of 1.65 eq of DIC/HOBt. After 19 hours, the reaction completion was checked by Ninhydrin tests. Negative tests were obtained for the chloro derivative (reaction complete).
Positive tests were obtained for the bromo derivative (reaction not complete).

Despite a positive coupling test on the bromo derivative, the coupling was stopped. The peptide-resin was washed in both experiments with DMF (3×5 mL).

Following the positive coupling test on the experiment using the 3-bromopropionic acid (Example 3.2.2) a micro-cleavage was performed in order to determine the HPLC purity of linear peptide.

### Micro-Cleavage

The micro-cleavage was carried out according to the conditions described in the process according to Example 3 (see, e.g., section 3.2.3). 40 mg of protected peptide-resin were suspended in a TFA/TIS/EDT/PhOH/H₂O (90/3/2/1/4) (v/v/v/v/v) mixture (424 μL) and stirred for 2 hours at room temperature. The reaction mixture was then filtered off and the resin was washed with TFA. The TFA mixture was poured onto dry Et₂O (4.24 ml); no precipitation of the crude peptide was obtained. The mixture was concentrated to dryness and the residue was dissolved in H₂O/CH₃CN (50/50) at a concentration of 1 mg/mL. This solution was analysed by HPLC and LC/MS. No peak corresponding to crude linear bromo peptide was present in the chromatogram purity of 0%).

The main compounds identified by LC/MS were:
H-D-Trp-Ile-allolle-Asn-hCy-OH corresponding to uncoupled bromopropionic acid and N-MeOrn deletion.
H-D-Trp-Ile-Ile-alloIle-Asn-hCy-N-MeOrn-OH or H-DTrp-Ile-alloIle-alloIle-Asn-hCy-N-MeOrn-OH corresponding to uncoupled bromopropionic acid and addition of either isoleucine or allo-isoleucine.
H-D-Trp-Ile-alloIle-hCy-N-MeOrn-OH corresponding to uncoupled bromopropionic acid.

These results demonstrate that the coupling of the 3-bromopropionic acid was not achieved for this experiment. Following this result, only the experiment using 3-chloropropionic acid (experiment according to Example 3.2.1) was engaged in the next steps (cysteine deprotection and cyclization).

Deprotection of homo-Cysteine and Cyclization

The peptide-resin was washed with DCM (3×5 mL) and the mmt group was deprotected by five repeated cycles (5×10 min) with 4 mL of a TFA/EDT/DCM (2/3/95 v/v/v) mixture.

After the deprotection, washings were performed with DCM (3×5 mL) and DMF (3×5 mL). Cyclization was performed on the resin by adding 5 mL of tetramethylguanidine solution (1% in DMF) and stirring during 8 hours at room temperature. After cyclization, the cyclic peptide-resin was washed with DMF (3×6 mL), DCM (3×6 mL), MeOH (5×6 mL) and dried under vacuum. Yield 15.1%.

### Cleavage

Cleavage from resin was performed according to the general conditions described in section 3.2.3. 297 mg of protected cyclic peptide-resin were suspended in a TFA/TIS/EDT/PhOH/H₂O (90/3/2/1/4) (v/v/v/v/v) mixture (3.15 mL) and the reaction mixture was stirred at room temperature during 2 hours. The reaction mixture was then filtered off and the resin was washed with TFA (210 μL). The TFA mixture was poured onto dry Et₂O (31.5 mL); as the experiments through the first pathway, no precipitation of the crude peptide was obtained. The mixture containing the crude peptide was concentrated to dryness on a rotary evaporator. The concentration residue was dissolved in H₂O/CH₃CN (50/50 v/v) and freeze-dried. After freeze-drying, a very low quantity of the powder was obtained (ar. 1-3 mg). Purity 10.4%.

### 3.5.4. Summary of the Experiments According to the Different Processes of Example 3

The aim of these experiments was to evaluate a different manufacturing process to synthesize the crude Barusiban by using two different pathways: one pathway in which all coupling reactions are performed for 2 hours and another pathway by executing the coupling test (Kaiser for completeness). Following table (Table 4) summarizes the assembly data obtained for both pathways:

TABLE 4
Summary table of assembly data obtained
for the first and the second pathways.

| | First pathway | | Second pathway | | | |
|---|---|---|---|---|---|---|
| | (Example 3.1) | | (Example 3.2) | | | |
| | 3.1.1 | 3.1.2 | 3.2.1 | | 3.2.2 | |
| Reference | (Cl) | (Br) | (Cl) | | (Br) | |
| AA coupling times | | | | | | |
| Fmoc-hCy(mmt)-OH | 2 h | 2 h | 4 | h | 4 | h |
| Fmoc-Asn(Tr)-OH | 2 h | 2 h | 2 | h* | 2 | h* |
| F/noc-alloIle-OH | 2 h | 2 h | 2 | h* | 2 | h* |
| Fmoc-Ile-OH | 2 h | 2 h | 2 | h* | 2 | h* |
| Fmoc-D-Trp(Boc)-OH | 2 h | 2 h | 2 | h* | 2 | h* |

| 3-halo propionic acid | 2 h | 2 h | 46 h 15** | 46 h 15** |
|---|---|---|---|---|
| Assembling yield | % | 13.3% | | — |

| | | | | |
|---|---|---|---|---|
| | 8.3% | | 15.1% | — |
| HPLC purity of the | 11.8% | 1.8% | 10.4% | — |
| crude on System I | | | | |

*The coupling was complete after 1 h but the reaction was stirred during 2 h
**Double coupling (DC) performed: 27 h 15 + 19 h (DC)

The HPLC purity of the crudes is very low for both pathways (Examples 3.1 and 3.2). Nevertheless the experiments using 3-chloropropionic acid lead to crude purities around 10% whereas the use of 3-bromopropionic leads to a purity of only 2%. It is also to mention that no crude was obtained in experiment according to Example 3.2.2 by using 3-bromopropionic acid even by controlling the coupling reactions. The data show that coupling of Fmoc-hCy(mmt)-OH requires more than 2 hours. The coupling of the 3-halopropionic acid requires also more than 2 hours; indeed it is observed in the second pathway that coupling was not achieved after 2 hours and double coupling was required to reach a negative coupling test on the chloro derivative. Furthermore the bromo derivative could not be incorporated even after double coupling. Crude Barusiban could be obtained according to the process according to Example 3 with low assembly yields (between 0 and 15%) and low HPLC purities (between 0 and 12%).

### 4. Example 4: Comparison Between Processes (Examples 1 and 2 vs Example 3)

The sections above described the synthesis of crude Barusiban according to the manufacturing process according to Examples 1 and 2 and according to the process according to Example 3 (3.1 and 3.2). For each processes (Examples 1 and 2 vs Example 3), it was studied whether 3 chloropropionic acid (Examples 2, 3.1.1 and 3.2.1) or 3 bromopropionic acid (Examples 1, 3.1.2 and 3.2.2) was more suitable for the synthesis of Barusiban. The results obtained for both processes (Examples 1 and 2 vs Example 3) are compared in order to assess which of the methods is the most appropriate/suitable to synthesize the crude Barusiban.

### 4.1. General Data

The following table (Table 5) summarizes and compares the assembly data obtained on experiments in the processes according to Examples 1 and 2 and in the processes according to Example 3.

TABLE 5
Comparative table of assembly data for experiments in the processes according to
Examples 1 and 2 and Example 3.

| | | | Example 3 | | | |
|---|---|---|---|---|---|---|
| | | | Example 3.1 | | Example 3.2 | |
| | Examples 1 and 2 | | (1st pathway) | | (2nd pathway) | |
| | Chloro | Bromo | Chloro | Bromo | Chloro | Bromo |
| | (Ex. 2) | (Ex. 1) | (Ex. 3.1.1) | (Ex. 3.1.2) | (Ex. 3.2.1) | (Ex. 3.2.2) |
| P*-N-MeOrn-(Boc)-ol | 17 h | 17 h | 2 h | 2 h | 2 h | 2 h |
| Fmoc-hCy(P)-OH | 16 h | 19.5 h | 2 h | 2 h | 4 h | 4 h |
| AA coupling times | 2 h-3.5 h | 2 h-3 h | 2 h | 2 h | 2 h | 2 h |
| (Asn, allolle, Ile D-Trp) | | | | | | |
| 3-halo propionic acid | 2 h | 1 h 15 | 2 h | 2 h | 46 h 15** | 46 h 15** |
| Assembly yield of | 92.4% | 89.8% | 8.3% | 13.3% | 15.1% | Not |
| peptide-resin | (Linear) | (Linear) | (Cyclic) | (Cyclic) | (Cyclic) | detected |
| | 86.36% | 84.2%- | — | — | — | (Cyclic) |
| | | — | 11.8% | 1.8% | 10.4% | — |
| | | | | | | — |

*P stands for “protecting group”. Depending on the process, P may be Fmoc or NBS, mtt or trt, see Examples 1, 2 and 3
**46 h 15: the coupling of 21 h 15 followed by addition of 1.65 eq of DIC (6 h) + a double coupling performed (19 h)

### 4.2. Intermediate Conclusion

In general no significant difference was observed for the coupling times the Asn, allolle, Ile and D-Trp residues. The coupling of the 3-halopropionic acid seems difficult in the processes according to Example 3, and a double coupling was necessary. The reaction was complete for the 3-chloropropionic acid, but it was incomplete for the 3-bromopropionic acid, even after double coupling. The coupling time of the 3-haloproponic acid takes only less than 2 hours if the process according to Example 1, and no double coupling is required. By following the manufacturing process according to Example 3, it appears that the different crudes are obtained with HPLC purity between 0 and 12% and a yield between 0 and 15%. These results are much lower than the data from the process according to Example 1.

### 5. Example 5. Stability Study of the Starting Materials

### 5.1. Stability Study of Fmoc-N-MeOrn(Boc)-ol

The instability of the raw material Fmoc-N-MeOrn(Boc)-ol has been observed in solution in DMSO (conditions for NMR analysis). After 24 h, Fmoc-N-MeOrn(Boc)-ol was degraded and the presence of dibenzofulvene was detected. Consequently a stability study of Fmoc-N-MeOrn(Boc)-ol was conducted. The aim of this study is to check the stability of Fmoc-N-Me-L-Orn(Boc)-ol in various conditions in order to define its optimal use and storage conditions. Some experiments were conducted to assess the degradation of the Fmoc-N-Me-L-Orn(Boc)-ol in various different storage conditions.

The stability is studied as follows:
Fmoc-N-Me-L-Orn(Boc)-ol, powder, at 2-8° C., room temperature and 40° C.
Fmoc-N-Me-L-Orn(Boc)-ol, solution in DMF (545 g/L) at 2-8° C., room temperature and 40° C.
Fmoc-N-Me-L-Orn(Boc)-ol+DMAP, solution in DMF at room temperature (corresponding to the loading conditions in the process synthesis according to Example 3)
Fmoc-N-Me-L-Orn(Boc)-ol+Pyridine, solution in DMF at 60° C. (corresponding to the loading conditions in the process according to Examples 1 and 2)

### 5.1.1. Stability of the Powder Fmoc-N-Me-L-Orn(Boc)-ol

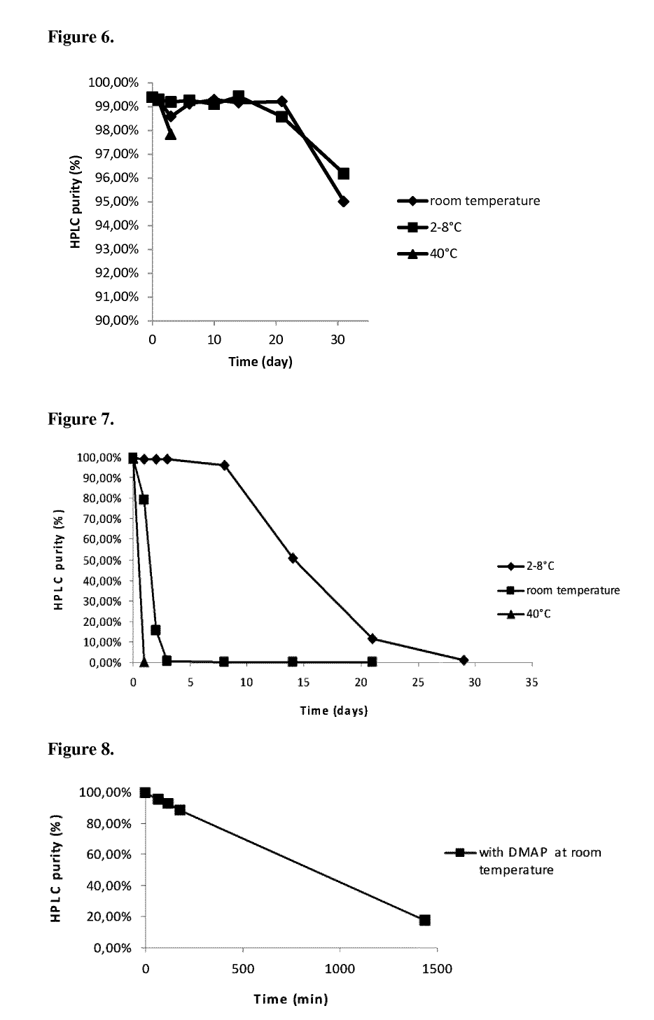

FIG. 6 represents the results obtained for the stability of the powder Fmoc-N-Me-L-Orn(Boc)-ol 2-8° C., room temperature and 40° C. Fmoc-N-Me-L-Orn(Boc)-ol is not stable at 40° C. as a powder. After 3 days, 1.55% of purity loss is observed (decrease from 99.26% to 97.84%, After 6 days, the powder turned into a glue with difficult solubilisation and making impossible the HPLC analysis. Fmoc-N-Me-L-Orn(Boc)-ol is stable as a solid at 2-8° C. and at room temperature for at least 14 days. After this period, a slight degradation is observed. In conclusion the powder Fmoc-N-Me-L-Orn(Boc)-ol can be stored at 2-8° C. for at least 14 days without any risk of degradation.

### 5.1.2. Stability of Fmoc-N-Me-L-Orn(Boc)-ol, Solution in DMF at Different Temperatures

FIG. 7 represents the results obtained for the stability of Fmoc-N-Me-L-Orn(Boc)-ol, solution in DMF (545 g/L) at 2-8° C., room temperature and 40° C. The purity of Fmoc-N-Me-L-Orn(Boc)-ol stored in DMF decreases rapidly over the time at room temperature and at 40° C. The product is completely degraded after 1 day at 40° C. and after 3 days at room temperature Storage at 2-8° C. allows slowing down the degradation. Purity is maintaining during 3 days. 50% degradation is observed after 2 weeks and complete decomposition after one month. The compound degrades by Fmoc deprotection leading to unprotected H-N-Me-L-Orn(Boc)-ol and dibenzofulvene. In contrast, for the storage at 2-8° C., no significant degradation of Fmoc-N-Me-L-Orn(Boc)-ol is observed in DMF up to 3 days. The purity of Fmoc-N-Me-L-Orn(Boc)-ol is decreased of 0.3% after 3 days and of 3% after 8 days. After 2 weeks, the purity of Fmoc-N-Me-L-Orn(Boc)-ol is decreased by half. To conclude Fmoc-N-Me-L-Orn(Boc)-ol in DMF can be stored at 2-8° C. for 3 days.

### 5.1.3. Stability of Fmoc-N-Me-L-Orn(Boc)-ol+DMAP, Solution in DMF at Room Temperature

FIG. 8 represents the results obtained for the stability of Fmoc-N-Me-L-Orn(Boc)-ol+DMAP solution in DMF (loading conditions in the process according to Example 3). The HPLC profiles were obtained. As expected, the degradation of Fmoc-N-Me-L-Orn(Boc)-ol is increased with the presence of DMAP. After 2 hours, the HPLC purity of Fmoc-N-Me-L-Orn(Boc)-ol is decreased by 7.5% and after 3 hours the HPLC purity of Fmoc-N-Me-L-Orn(Boc)-ol is 88,24%. After 24 hours the purity of Fmoc-N-Me-L-Orn(Boc)-ol is very low (17.71%). In conclusion, Fmoc-N-Me-L-Orn(Boc)-ol+DMAP, solution in DMF is not stable for a long period in the loading conditions according to the process of Example 3. Some degradation is already observed after 2 hours at room temperature; the material can potentially decompose partially during the loading reaction.

### 5.1.4. Stability of Fmoc-N-Me-L-Orn(Boc)-ol+Pyridine, Solution in DMF at 60° C.

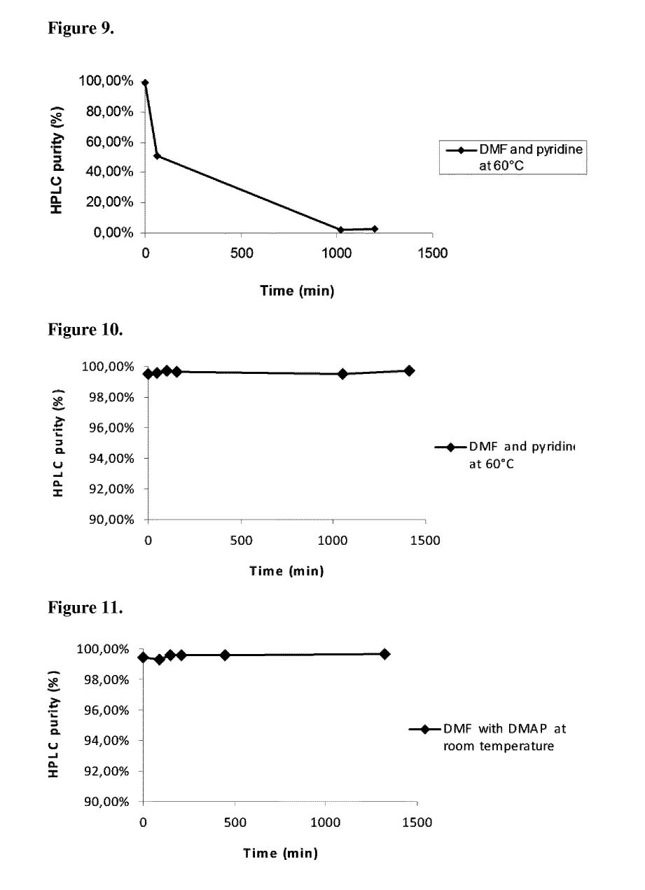

FIG. 9 represents the results obtained for the stability of Fmoc-N-Me-L-Orn(Boc)-ol+Pyridine, solution in DMF at 60° C. (loading conditions according to the processes of Examples 1 and 2). In the conditions of the loading step used in the manufacture process according to Examples 1 and 2, Fmoc-N-Me-L-Orn(Boc)-ol degrades very quickly; 50% loss of purity is observed after 1 hour and almost complete degradation 17 hours. To conclude, Fmoc-N-Me-L-Orn(Boc)-ol is not stable in the loading conditions according to the processes of Examples 1 and 2, as the degradation is already started after 1 hour and the compound is not stable all over the loading time (17 hours).

### 5.2. Stability Study of o-NBS-N-Me-Orn(Boc)-ol

A stability study of o-NBS-N-Me-Orn(Boc)-ol was conducted. The aim of this study is to check the stability of o-NBS-N-Me-L-Orn(Boc)-ol in various conditions in order to define its optimal use. Some experiments were conducted to assess the degradation of the o-NBS-N-Me-L-Orn(Boc)-ol in various loading conditions.

The stability is studied as follows:
o-NBS-N-Me-L-Orn(Boc)-ol+DMAP, solution in DMF at room temperature (corresponding to the loading conditions according to the process as described in Example 3)
o-NBS-N-Me-L-Orn(Boc)-ol+Pyridine, solution in DMF at 60° C. (corresponding to the loading conditions in the process as described in Examples 1 and 2)

### 5.2.1. Stability of o-NBS-N-MeOrn(Boc)-ol+Pyridine, Solution in DMF at 60° C.

FIG. 10 represents the results obtained for the stability of o-NBS-N-Me-L-Orn(Boc)-ol+Pyridine solution in DMF (loading conditions in the process according to Examples 1 and 2). In the conditions of the loading step used in the process according to Examples 1 and 2, o-NBS-N-Me-L-Orn(Boc)-ol is stable. After 24 hours, the purity of o-NBS-N-Me-L-Orn(Boc)-ol is 99,68%. In conclusion, o-NBS-N-Me-L-Orn(Boc)-ol is stable in the loading conditions of the processes according to Examples 1 and 2.

### 5.2.2. Stability of o-NBS-N-Me-Orn(Boc)-ol+DMAP Solution in DMF at Room Temperature

FIG. 11 represents the results obtained for the stability of o-NBS-N-Me-L-Orn(Boc)-ol+DMAP solution in DMF (loading conditions in the processes according to Example 3). In the conditions of the loading step used in the processes according to Example 3, o-NBS-N-Me-L-Orn(Boc)-ol is stable. After 22 hours, the purity of o-NBS-N-Me-L-Orn(Boc)-ol is 99.62%. In conclusion, o-NBS-N-Me-L-Orn(Boc)-ol is stable in the loading conditions according to the processes of Example 3.

### 5.2.3. Conclusion of the Stability Study

The Fmoc-N-Me-Orn(Boc)-ol used in the processes according to Example 3 is a sensitive material which degrades rapidly in the conditions used for the loading step. On the other hand, the o-NBS protected material remains stable whatever the loading conditions (processes according to Examples 1, 2 or 3).

### 6. General Conclusion

Examples 1 and 2 describe the process of manufacture of Barusiban according to the present invention. This process is compared to a different process of manufacture of Barusiban (Example 3).

In total five experiments were performed by using either 3-chloropropionic acid or 3-bromopropionic acid (Examples 1, 2, 3.1.1, 3.1.2, 3.2.1 and 3.2.2).
The Fmoc-N-Me-Orn(Boc)-ol used in the processes according to Example 3 is a sensitive material which degrades rapidly in the conditions used for the loading step: the rapid degradation might impact the loading yield.
On the other hand the o-NBS protected material remains stable whatever the loading conditions (Examples 1, 2 or 3); this derivative is more adapted as it shows a better stability in basic conditions (pyridine or DMAP).
Whatever the reagent used for the last coupling in the processes according to Example 3 (3-chloro/3-bromopropionic acid), the coupling was difficult and a double coupling was necessary to reach completion. The reaction was complete with 3-chloropropionic acid and not complete with 3-bromopropionic acid. The use of 3-chloropropionic acid is possible in the process according to the present invention (Example 2).
Crudes on the processes according to Example 3 are obtained with HPLC purity between 0 and 12% and a yield between 0 and 15%. These results are much lower than the current data according to the process according to Examples 1 and 2, leading to 45% HPLC purity and 90% yield.

Based on these conclusions, best results are definitely obtained with the process according to Examples 1 and 2, particularly with the process according to Example 1. The Barusiban manufacturing process according to Examples 1 and 2 shows a better robustness and is well adapted to obtain correct purity and yield.

### Items of the Present Invention

1. A solid phase process for preparing a compound having the formula c[AA₁-AA₆]-AA₇-OH, or a pharmaceutically acceptable salt or solvate thereof, wherein AA₁ is propionic acid, AA₂ is AA_{b}, AA₃ is Ile, AA₄ is AA_{d}, AA₅ is Asn, AA₆ is hCy, and AA₇ is —NR—CHQ-CH₂OH, wherein R is CH₃ or C₂H₅, Q is (CH₂)ₙ—NH₂, wherein n is 2, 3, or 4, the process comprising the steps of:
a) reacting protected (P₅;P₃)AA₇ with a resin to provide (P₅;P₃)AA₇- [symbol] wherein AA₇ as added to the resin during the synthesis in step a) is (P₅)NR-CHQ′-CH₂OH;
b) stepwise lengthening (P₅;P₃)AA₇- [symbol] to provide (P₇)AA₂AA₃AA₄(P₁)AA₅(Trt)AA₆-(P₃)AA₇- [symbol] ;
c) reacting X-AA₁ (wherein X is an halogen atom selected from F, Cl, Br and I) with (P₇)AA₂AA₃AA₄(P₁)AA₅(Trt)AA₆-(P₃)AA₇- [symbol] to provide X-AA₁(P₇)AA₂AA₃AA₄(P₁)AA₅(Trt)AA₆-(P₃)AA₇- [symbol] ;
d) carrying out a cleavage and deprotection step to provide X-AA₁AA₂AA₃AA₄AA₅AA₆AA₇-ol; and
e) cyclizing X-AA₁AA₂AA₃AA₄AA₅AA₆AA₇-ol, obtaining the cyclic peptide: c[AA₁-AA₆]-AA₇-ol wherein AA₁ and AA₆ are linked through a thiol from the AA₆ homocysteine,
wherein:
P₁, P₅, and P₇ are protecting groups,
AA_{b} is a D-aromatic α-amino acid;
AA_{d} is an aliphatic α-amino acid;
X is a halogen residue;
R is CH₃ or C₂H₅;
Q′ is (CH2)ₙ—NP₃P₄;
n is 2, 3, or 4;
P₃ and P₄ are independently H or an amino-acid protecting group, which may be the same or different from each other and which may the same or different to P₁.

2. The solid phase process according to item 1, wherein the resin is 2-CTC.

3. The solid phase process according to one or more of the preceding items, wherein P₅ is

NBS, and/or wherein P₃ and P₇ are both Boc and/or wherein P₁ is Trt.

4. The solid phase process according to one or more of the preceding items, wherein X is Br or Cl, preferably Br.

5. The solid phase process according to one or more of the preceding items, wherein n is 3 and/or R is CH₃, and/or wherein preferably P₄ is H and/or wherein preferably P₃ is Boc.

6. The solid phase process according to one or more of the preceding items, wherein reacting step c) is carried out with DIC as coupling agent in the presence of DCM.

7. The solid phase process according to one or more of the preceding items wherein AA₂ (AA_{b}) is D-Trp, and/or AA₄ (AA_{d}) is Allolle, and/or AA₇ is N-Me-Orn-ol.

8. The solid phase process according to one or more of the preceding items, wherein the process optionally comprises one or more purification steps, preferably one or more purification steps after step i) (namely after the cleavage and deprotection step) and/or one or more purification steps after step j) (namely after the cyclization step).

9. An intermediate suitable for forming a peptide having pharmaceutical properties, which has the formula:

X—(CH₂)₂—CO—NH-AA_{b}-Ile-AA_{d}-Asn(P₁)-hCy(P₂)—NR-CHQ′-CH₂OW

wherein

AA_{b} is a D-aromatic α-amino acid;

AA_{d} is an aliphatic α-amino acid;

X is a halogen residue; P₁ is a protecting group

P₂ is Trt

R is CH₃ or C₂H₅;

Q′ is (CH₂)ₙ—NP₃P₄;

n is 2, 3, or 4;

P₃ and P₄ are independently H or an amino-acid protecting group, which may be the same or different from each other and which may the same or different to P₁ and/or P₂; and

W is H, a protecting group or a resin.

10. The intermediate according to item 9, wherein W is a resin, wherein preferably the resin is CTC.

11. The intermediate according to item 9-10, wherein n is 3 and/or R is CH₃.

12. The intermediate according to any of items 9-11, wherein P₁ is Trt.

13. The intermediate according to any of items 9-12, wherein P₄is H and/or P₃ is Boc.

14. The intermediate according to any of items 9-13, wherein AA_{b} is D-Trp, and/or wherein AA_{d} is allolle.

15. The intermediate according to any of items 9-14, wherein X is Br or Cl, preferably Br.

1. A solid phase process for preparing a compound having the formula c[AA₁-AA₆]-AA₇-OH, wherein AA₁ and AA₆ are linked through a thiol from the AA₆ homocysteine, or a pharmaceutically acceptable salt or solvate thereof, wherein AA₁ is propionic acid, AA₂ is AA_{b}, AA₃ is Ile, AA₄ is AA_{d}, AA_{S} is Asn, AA₆ is hCy, and AA₇ is —NR-CHQ-CH₂OH, Q is (CH₂)ₙ—NH₂, the process comprising the steps of:
a) reacting protected (P₅P₃ P₄)AA₇ with a resin to provide (P₅P₃P₄)AA₇- [symbol] wherein protected (P₅P₃P₄) AA₇ as added to the resin during the synthesis in step a) is (P₅)NR-CHQ′-CH₂OH;
b) stepwise lengthening (P₅P₃P₄)AA₇- [symbol] to provide (P₇)AA₂AA₃AA₄(P₁)AA₅(Trt)AA₆-(P₃P₄)AA₇- [symbol] ;
c) reacting X-AA₁ (wherein X is an halogen atom selected from F, Cl, Br and I) with (P₇)AA₂AA₃AA₄(P₁)AA₅(Trt)AA₆-(P₃P ₄)AA₇- [symbol] to provide X-AA₁(P₇)AA₂AA₃AA₄(P₁)AA₅(Trt)AA₆-(P₃P₄)AA₇- [symbol] ;
d) carrying out a cleavage and deprotection step to provide X-AA₁AA₂AA₃AA₄AA₅AA₆AA₇-ol; and
e) cyclizing X-AA₁AA₂AA₃AA₄AA₅AA₆AA₇-ol, obtaining the cyclic peptide: c[AA₁-AA₆]-AA₇-ol wherein AA₁ and AA₆ are linked through a thiol from the AA₆ homocysteine,
wherein:
P₁, P₅, and P₇ are protecting groups,
AA_{b} is a D-aromatic α-amino acid;
AA_{d} is an aliphatic α-amino acid;
X is a halogen residue;
R is CH₃ or C₂H₅;
Q′ is (CH2)ₙ—NP₃P₄;
n is 2, 3, or 4;
P₃ and P₄ are independently H or an amino-acid protecting group, which may be the same or different from each other and which may the same or different to P₁.

2. The solid phase process according to claim 1, wherein the resin is 2-CTC.

3. The solid phase process according to one or more of the preceding claims, wherein P₅ is NBS, and/or wherein P₃ and P₇ are both Boc and/or wherein P₁ is Trt.

4. The solid phase process according to one or more of the preceding claims, wherein X is Br or Cl, preferably Br.

5. The solid phase process according to one or more of the preceding claims, wherein n is 3 and/or R is CH₃, and/or wherein preferably P₄ is H and/or wherein preferably P₃ is Boc.

6. The solid phase process according to one or more of the preceding claims, wherein reacting step c) is carried out with DIC as coupling agent in the presence of DCM.

7. The solid phase process according to one or more of the preceding claims wherein AA₂ (AA_{b}) is D-Trp, and/or AA₄ (AA_{d}) is Allolle, and/or AA₇ is N-Me-Orn-ol.

8. The solid phase process according to one or more of the preceding claims, wherein the process optionally comprises one or more purification steps, preferably one or more purification steps after step i) (namely after the cleavage and deprotection step) and/or one or more purification steps after step j) (namely after the cyclization step).

9. An intermediate suitable for forming a peptide having pharmaceutical properties, which has the formula:
X—(CH₂)₂—CO—NH-AA_{b}-Ile-AA_{d}-Asn(P₁)-hCy(P₂)—NR-CHQ′-CH₂OW
wherein
AA_{b} is a D-aromatic α-amino acid;
AA_{d} is an aliphatic α-amino acid;
X is a halogen residue;
P₁ is a protecting group
P₂ is Trt
R is CH₃ or C₂H₅;
Q′ is (CH₂)ₙ—NP₃P₄;
n is 2, 3, or 4;
P₃ and P₄ are independently H or an amino-acid protecting group, which may be the same or different from each other and which may the same or different to P₁ and/or P₂; and
W is H, a protecting group or a resin.

10. The intermediate according to claim 9, wherein W is a resin, wherein preferably the resin is CTC.

11. The intermediate according to claim 9-10, wherein n is 3 and/or R is CH₃.

12. The intermediate according to any of claims 9-11, wherein P₁ is Trt.

13. The intermediate according to any of claims 9-12, wherein P₄is H and/or P₃ is Boc.

14. The intermediate according to any of claims 9-13, wherein AA_{b} is D-Trp, and/or wherein AA_{d} is allolle.

15. The intermediate according to any of claims 9-14, wherein X is Br or Cl, preferably Br.
